(19) United States
(12) Patent Application Publication
(10) Pub. No.: US 2018/0009859 A1
(43) Pub. Date: Jan. 11, 2018

(54) PHARMACOLOGICAL MODULATORS OF GABAA RECEPTORS AND THEIR USE

(71) Applicant: THE JOHNS HOPKINS UNIVERSITY, Baltimore, MD (US)

(72) Inventors: Frank Bosmans, Annapolis, MD (US); Pierre Edouard Bougis, Marseille (FR)

(21) Appl. No.: 15/544,030

(22) PCT Filed: Jan. 14, 2016
(86) PCT No.: PCT/US2016/013318

§ 371 Date: Jul. 17, 2017

Related U.S. Application Data
(60) Provisional application No. 62/103,669, filed on Jan. 15, 2015.

(51) Int. Cl. C07K 14/47; A61K 49/00; A61K 38/00

(52) U.S. Cl. CPC C07K 14/4702; A61K 49/0056; A61K 38/00

(57) ABSTRACT

The present invention provides polypeptides and their variants and derivatives which modulate GABA_{A} receptor function at nanomolar quantities by binding to the α+/β− subunit interface, as well as methods of making and using the same. The inventive peptides are useful for the study of neurological disease and function and can have therapeutic applications.

### REFERENCE TO RELATED APPLICATIONS

This application claims the benefit of U.S. Provisional Patent Application No. 62/103,669, filed on Jan. 15, 2015, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Dec. 28, 2015, is named P13167-02_ST25.txt and is 12,588 bytes in size.

### BACKGROUND OF THE INVENTION

Ionotropic γ-aminobutyric acid type A (GABA_{A}) receptors are found primarily in the central nervous system (CNS) where they mediate inhibitory post-synaptic transmission by increasing Cl⁻ conductance into the cell. However, GABA_{A} receptor activation can be excitatory in neonatal neurons and dorsal root ganglia where intracellular Cl⁻ concentrations are high and receptor activation causes Cl⁻ efflux which in turn, depolarizes the cell. When the balance between excitatory and inhibitory GABA_{A} receptor activity is shifted due to abnormal function, clinical phenotypes such as epilepsy, schizophrenia, and chronic pain can occur. As such, GABA_{A} receptors are targeted by various drugs including barbiturates, benzodiazepines, and anesthetics.

GABA_{A} receptors belong to the pentameric Cys-loop superfamily of ligand-gated ion channel receptors which also encompasses the nicotinic acetylcholine (nAChRs), glycine (GlyR), and serotonin (5-HT3) receptors. The numerous subunit isoforms (α1-6, β1-3, γ1-3, δ, ε, π, θ, and ρ1-3) that can make up a GABA_{A} receptor create multiple potential structural arrangements. In general, each subunit consists of four trans-membrane domains (TM1-4), in which TM2 delineates the axially positioned Cl⁻ channel. Molecules can interact with various regions within one or more subunits, resulting in a complex pharmacological landscape. For example, GABA and muscimol bind at the extracellular interface between a β- and α-subunit (β+/α−) whereas benzodiazepines require both the α- and γ2-subunit to be pharmacologically active. Conversely, anesthetics such as propofol most likely position themselves in transmembrane inter-subunit pockets. So far, picrotoxin (PTX) is the only well-documented naturally-occurring plant toxin that is known to block the pore of GABA_{A} receptors and is experimentally used as a chemoconvulsant to induce epileptic seizures. This is in contrast to nAChRs, where molecules isolated from plant extracts, snake and cone snail venoms have been used extensively to probe the structural and functional properties of these receptors.

### SUMMARY OF THE INVENTION

The present inventors discovered two toxins present in Costa Rican coral snake venom, named MmTX1 and MmTX2, which bind to GABA_{A} receptors at nanomolar concentrations. Studies with recombinant and synthetic toxin variants on hippocampal neurons and cells expressing common receptor compositions show that MmTX1 and MmTX2 and polypeptide homologs and variants thereof, can potentiate GABA_{A} receptor opening and accelerate desensitization when an agonist is present, possibly by interacting with the α+/β− interface.

In accordance with an embodiment, the present invention provides polypeptides having GABA_{A} modulating activity.

In accordance with an embodiment, the present invention provides polypeptides having GABA_{A} modulating activity comprising the following amino acid sequence a) LTCKTCPFTTCPNSESCPGGQSICYQRKWEEHRGERIERRCVANCPAFGSHDTSLLCC TRDNCN (MmTX1) (SEQ ID NO: 1), b) a functional fragment of a); c) a functional homolog of a) or b) or functional fragment thereof; and d) a fusion polypeptide comprising an amino acid sequence of any of a) to c).

In accordance with an embodiment, the present invention provides polypeptides having GABA_{A} modulating activity comprising the following amino acid sequence a) LTCKTCPFTTCPNSESCPGGQSICYQRKWEEHHGERIERRCVANCPAFGSHDTSLLCC TRDNCN (MmTX2) (SEQ ID NO: 2), b) a functional fragment of a); c) a functional homolog of a) or b) or functional fragment thereof; and d) a fusion polypeptide comprising an amino acid sequence of any of a) to c).

In yet another embodiment, the present invention provides polypeptides having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His Arg Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 6), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His His Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 7), b) a functional fragment of a); c) a functional homolog of a) or b) or functional fragment thereof; and d) a fusion polypeptide comprising an amino acid sequence of any of a) to c).

In accordance with a further embodiment, the present invention provides polypeptides having GABA_{A} modulating activity comprising the following amino acid sequence: a) Leu Thr Cys Lys Thr Cys Pro Phe Thr Thr Cys Pro Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 8), b) a functional fragment of a); c) a functional homolog of a) or b) or functional fragment thereof; and d) a fusion polypeptide comprising an amino acid sequence of any of a) to c).

In accordance with still another embodiment, the present invention provides polypeptides having GABA_{A} modulating activity comprising the following amino acid sequence: a) Met Lys Cys Leu Ile Cys Pro Phe Thr Thr Cys Ser Gln Ser Glu Ser Cys Pro Gly Gly Gln Ser Ile Cys Phe Gln Arg Lys Phe Asp Asp Arg His Gly Asp Arg Ile Glu Arg Gly Cys Ala Val Thr Cys Pro Pro Phe Gly Ser His Asp Thr Ile Phe Cys Cys Ser Thr Asn Asp Cys Asn (SEQ ID NO: 9), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Ile Glu Cys His Asn Cys Pro Phe Thr Thr Cys Gly Asn Ser Glu Ser Cys Pro Gly Gly Gln Ser Ile Cys Val Gln Arg Lys Leu Glu Glu Lys Lys Gly Glu Arg Ile Glu Arg Ser Cys Thr Asp Gly Cys Pro Gly Phe Gly Ser His Asp Thr Val Glu Cys Cys Arg Ile Ala Arg Cys Asn (SEQ ID NO: 10), b) a functional fragment of a); c) a functional homolog of a) or b) or functional fragment thereof; and d) a fusion polypeptide comprising an amino acid sequence of any of a) to c).

In accordance with yet another embodiment, the present invention provides polypeptides having GABA_{A} modulating activity comprising the following amino acid sequence: a) Arg Gln Cys Tyr Thr Cys Pro Phe Thr Thr Cys His Asn Ser Glu Ser Cys Pro Gly Gly Gln Ser Ile Cys Tyr Gln Arg Lys Tyr Glu Glu His Arg Gly Glu Arg Ile Glu Arg Lys Cys Ser Leu Ser Cys Pro Ser Phe Gly Ser His Asp Thr Leu Leu Cys Cys Ala Arg Pro Lys Cys Asn (SEQ ID NO: 11), b) a functional fragment of a); c) a functional homolog of a) or b) or functional fragment thereof; and d) a fusion polypeptide comprising an amino acid sequence of any of a) to c).

In accordance with a further embodiment, the present invention provides polypeptides having GABA_{A} modulating activity comprising the following amino acid sequence: a) Phe Arg Cys Phe Arg Cys Pro Phe Thr Thr Cys Asn Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Lys Arg Glu Glu Cys Asn (SEQ ID NO: 12), b) a functional fragment of a); c) a functional homolog of a) or b) or functional fragment thereof; and d) a fusion polypeptide comprising an amino acid sequence of any of a) to c).

In accordance with still another embodiment, the present invention provides polypeptides having GABA_{A} modulating activity comprising the following amino acid sequence: a) Leu Ser Cys Asn Thr Cys Pro Phe Thr Thr Cys Gln Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 13), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Leu Leu Cys Lys Thr Cys Pro Phe Thr Thr Cys Pro Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 14), b) a functional fragment of a); c) a functional homolog of a) or b) or functional fragment thereof; and d) a fusion polypeptide comprising an amino acid sequence of any of a) to c).

In accordance with an embodiment, the present invention provides a polypeptide having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His Arg Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 6); b) a functional fragment of a); c) a functional homolog of a) or b) or functional fragment thereof; and d) a fusion polypeptide comprising an amino acid sequence of any of a) to c).

In accordance with an embodiment, the present invention provides a polypeptide having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His His Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 7); b) a functional fragment of a); c) a functional homolog of a) or b) or functional fragment thereof; and d) a fusion polypeptide comprising an amino acid sequence of any of a) to c).

In accordance with an embodiment, the present invention provides one or more nucleic acid sequences encoding any of the polypeptides having GABA_{A} modulating activity or derivatives, homologues, analogues or mimetics thereof disclosed herein.

In accordance with another embodiment, the present invention provides a vector comprising one or more nucleic acid sequences encoding any of the polypeptides having GABA_{A} modulating activity or derivatives, homologues, analogues or mimetics thereof disclosed herein.

In accordance with an embodiment, the present invention provides a composition comprising one or more polypeptides having GABA_{A} modulating activity described herein, and at least one or more biologically active agents.

In accordance with an embodiment, the present invention provides a composition comprising one or more polypeptides having GABA_{A} modulating activity described herein, and at least one or more imaging agents.

In accordance with an embodiment, the present invention provides methods for modulating GABA_{A} receptors in a cell or population of cells expressing the GABA_{A} receptor comprising contacting the cell or population of cells with an effective amount of the inventive polypeptides described herein.

In accordance with an embodiment, the present invention provides methods for modulating GABA_{A} receptors in a subject suffering from a neurological disorder comprising administering to the subject a composition comprising one or more polypeptides having GABA_{A} modulating activity described herein, and at least one or more biologically active agents.

### BRIEF DESCRIPTION OF THE DRAWINGS

FIGS. 1A-1C: Native and recombinant MmTX1 and MmTX2 are equally potent. 1A, Picture of the Costa Rican coral snake Micrurus mipartitus. 1B, Primary amino acid sequences of MmTX1 and MmTX2. Note the different residue at position 33 (grey background). 1C, Competition experiments for the binding of ¹²⁵I-rMmTX2 to SPMs with WT MmTX1, rMmTX1, rMmTX2, and the ³³Ser mutant (rMmTXSer33). Lines represent a nonlinear fit with constrained ¹²⁵I-rMmTX2-parameters (K_{d}=0.51 nM and assay concentration 0.2 nM). Note that WT MmTX1, rMmTX1, and rMmTX2 are equally potent in displacing ¹²⁵I-rMmTX2 whereas rMmTXSer33 is ineffective.

FIGS. 2A-2D: Binding kinetics of rMmTX2 to SPMs. 2A, Association kinetics of ¹²⁵I-rMmTX2 binding to SPMs. Shown are the association curves for three ligand concentrations (1, 2, and 4 nM). Lines represent a nonlinear fit with a one-phase exponential association curve providing pseudo-first order association constants (k_{obs.}) that are used in panel 2B. 2B, Figure represents the k_{obs} linear variation with 1, 2, and 4 nM ligand concentrations. Slope represents the association kinetic constant (k₊₁; shown in inset). 2C, Dissociation kinetics of ¹²⁵I-rMmTX2 from SPMs. Once the initial toxin binding is achieved, the addition of a 1000-fold excess of unlabeled rMmTX2 reveals the time-course of toxin dissociation. Line represents a nonlinear fit with a one-phase exponential decay providing the shown rate constant (k₋₁) and a t1/2 of 478s (˜8 min). 2D, Saturation binding isotherm of ¹²⁵I-rMmTX2 to SPMs. Nonspecific binding as determined in the presence of a 1000-fold excess of unlabeled rMmTX2 has been subtracted. Line represents a nonlinear fit with one class of non-interacting binding sites giving an equilibrium binding constant (K_{d}) of 0.51±0.09 nM and a maximum specific binding (Bₘₐₓ) of 264±14 fmol/mg protein.

FIGS. 3A-3C: Pharmacological profiling of rMmTX2. 3A, Bar-plot of competition experiments between ¹²⁵I-rMmTX2 and a variety of ligands for binding to SPMs. The tested compounds are shown within the bar and their primary target is indicated on the X-axis. Concentrations used: all animal toxins 10 μM, (rMmTX2 0.1 μM), glycine 1 mM, glutamate 0.1 mM, atropine 1 μM, d-tubocurarine 10 μM. Diagram shows that none of the tested ligands competes with ¹²⁵I-rMmTX2 for binding to SPMs. 3B, Competition experiments of ¹²⁵I-rMmTX2 with a variety of GABA_{A} receptor modulators for binding to SPMs. Tested compounds are listed with their corresponding symbols. Lines represent a nonlinear fit with a sigmoidal dose-response curve (variable slope; Hill Slope) to provide the EC₅₀ values listed in the text. The only exception is picrotoxin (PTX) which increases the binding of ¹²⁵I-rMmTX2. 3C, Saturation binding isotherm of 125I-MmTX2 on SPMs in the absence or presence of 1 mM PTX.

FIG. 4: [³H]muscimol competition with rMmTX1 for binding to SPMs. rMmTX1 competes for binding to SPMs with [³H]muscimol. Line represents a nonlinear fit for the allosteric modulator with constrained [³H]muscimol parameters (K_{d}=1.75 nM and assay concentration 40 nM implying that both GABA binding sites are occupied).

FIGS. 5A-5C: Effect of rMmTX1 on hippocampal GABA_{A} receptor currents. 5A, Potentiation of the muscimol-induced GABA_{A} receptor current depends on rMmTX1 concentration. Currents were elicited with 5 μM muscimol without and with increasing rMmTX1 concentrations. Membrane holding voltage was −70 mV. 5B, Upper panel: currents elicited with 100 nM muscimol (left) followed by co-application with 100 nM rMmTX1 (middle), then without rMmTX1 (right). Middle panel: currents elicited with 300 nM muscimol. Lower panel: currents elicited with 50 μM muscimol. Bars represent the duration of sample application. 5C, Left: Dependence of the GABA_{A} receptor current amplitude on muscimol concentration. A Hill equation was fitted to the data points (filled circles) yielding an EC₅₀ of 4.1 μM and a Hill coefficient of 1.4. These data points and SEM were multiplied by the factor given in Table 3 yielding the rMmTX1-induced mean GABA_{A} current increase (filled squares). Right: Muscimol-dependent potentiating effect of rMmTX1 calculated as the toxin-induced current increase in relation to the total current amplitude. High muscimol concentrations abolish the potentiating effect with an IC₅₀ of 1.1 μM.

FIGS. 6A-6E: Effect of sMmTX1 on α1/β2/γ2 GABA_{A} receptor currents. 6A, A muscimol concentration of 3 μM activates α1β2γ2 GABA_{A} receptors expressed in HEK293 cells (left trace) held at a membrane voltage of −70 mV. When co-applying 300 nM sMmTX1, currents are potentiated and receptor desensitization tends to accelerate (middle trace). Partial toxin washout is shown on the right. 6B, Expression of the α1β2γ2 receptor in Xenopus oocytes held at a membrane voltage of −70 mV. Protocol consists of two 30s test applications of 5 μM muscimol separated by a 3 minute wash out period followed by 100 nM sMmTX1 application together with 5 μM muscimol. Under control conditions, receptors desensitize with a time constant of 48±5s (n=3). In the presence of 100 nM sMmTX1 the time constant is 25±2s (n=3). sMmTX1 washout is slow and after three minutes, only a fraction of the receptors can be reactivated. Moreover, available receptors activate more slowly when compared to control suggesting that toxin is still bound. 6C, Close-up of the red and green region indicated in panel 6B normalized to the control peak (indicated with an asterisk). 6D, When lowering the muscimol concentration to 0.3 μM, a similar effect of 100 nM sMmTX1 on receptor desensitization is observed. 6E, Similar to the α1β2γ2 receptor, the α1β2-containing receptor expressed in oocytes and held at −70 mV is sensitive to 100 nM sMmTX1.

FIGS. 7A-7G: sMmTX1 influences hippocampal network function. 7A, 300 nM muscimol does not have a detectable effect on the frequency of Ca²⁺ oscillations. 7B, sMmTX1 by itself does not affect the frequency of Ca²⁺ oscillations. 7C, when applied together with muscimol at 300 nM, sMmTX1 produces a short inhibition of network Ca²⁺ oscillations followed by a marked increase in spike frequency. 7D, Quantification of FIGS. 7A-7C represented as a percentage variation of the Ca²⁺ oscillation frequency with respect to cells perfused with extracellular solution (100%, dotted line). Data are presented as mean±SEM. 7E-7F, Representative traces of recordings of neurons perfused with ACSF (left) or muscimol-sMmTX1 (right), under voltage- and current-clamp configuration respectively (n=5). Zoomed-in areas indicated by the red rectangle in traces 7E and 7F are shown in red in the lower panel in each figure. 7G, Representative traces of recordings of neurons perfused with 100 μM PTX under voltage-(left) and current-(right) clamp configuration respectively (n=5). Zoomed-in areas indicated by the red rectangle are shown in red in the lower panel.

FIG. 8 shows the effect of 5 μM muscimol and 100 nM sMmTX1 on various GABA_{A} receptor subunit compositions. Expression of the various GABA_{A} receptor compositions in Xenopus laevis oocytes held at a membrane voltage of −70 mV. Protocol consists of two 30s test applications of 5 μM muscimol separated by a 3 minute wash out period followed by 100 nM sMmTX1 applications in concert with 5 μM muscimol. Currents could not be evoked from control (uninjected) oocytes. Close-up of the red and green region indicated in the lower panels are normalized to the control peak (indicated with an asterisk). Experiments with subunit compositions α2β3γ2, αβ2γ2, α5β3γ2, and α1β3 suggest an important role for the al subunit in determining GABA_{A} receptor susceptibility to sMmTX1. X-axis time scale is 90s. Representative example of 4 experiments (n=4) with each receptor composition are shown.

FIG. 9 depicts the effect of sMmTX1 on the α1EKβ2γ2 receptor mutant. Top panel shows an amino acid sequence alignment of rat α1-6 GABA_{A} receptor subunits indicating the pharmacologically important loop-C region. Number in italic indicates amino acid position in α1. Mutated residues in al are highlighted in red whereas the corresponding positions in α2-5 are displayed with a grey background. Accession numbers for sequences used are NP 899155.1, NP 001129251.1, NP 058765.3, NP 542154.3, NP 058991.1, and NP_068613.1 for α1-6, respectively. Middle panel shows expression of the α1EKβ2γ2 receptor in Xenopus oocytes held at a membrane voltage of −70 mV. Protocol consists of two 30s test applications of 5 μM muscimol separated by a 3 minute washout period followed by 100 nM sMmTX1 application together with 5 μM muscimol. In contrast to experiments with the WT al-subunit (FIG. 6C), the α1EKβ2γ2 receptor displays a rundown that is not present with the WT receptor (indicated by a solid line and extended by a dotted trend line). Strikingly, 100 nM sMmTX1 does not increase mutant receptor desensitization or significantly influences its response to 5 μM muscimol after toxin washout (n=4; representative examples shown). In the presence of 300 nM muscimol (lower panel), sMmTX1 still reduces the macroscopic conductance of the α1EKβ2γ2 receptor, yet toxin washout is markedly faster when compared to WT. Rundown is indicated by the same line as in the middle panel. X-axis time scale is 90s.

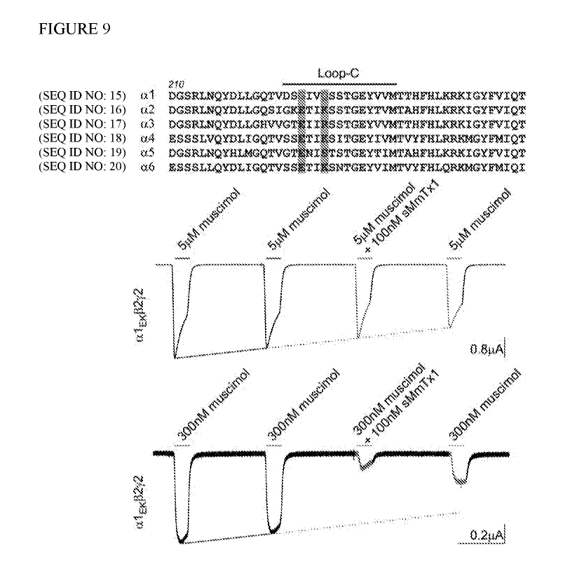

FIGS. 10A-10D show the effect of sMmTX1 on hippocampal neurons. A, Quantification of action potential (AP) properties without and in the presence of 100 nM sMmTX1 (see also FIG. 7). The frequency of APs is significantly increased by toxin treatment (p=0.0147, n=5) whereas amplitude and time-to-peak measurements do not reveal a significant change. Graphs are presented as paired changes for each experiment (absolute numbers, left Y-axis) and also as normalized grouped changes (%, right Y-axis). B, Continuous application of 100 nM sMmTX1 induces a decrease in currents elicited by 30 ms puffs of 300 nM muscimol. Control (Ctrl) trace (black) represents the average of currents recorded during 3 minutes under normal extracellular Ringer solution incubation. The sMmTX1 trace (red) is the result of averaging currents for 3 minutes after 3 minutes perfusion with 100 nM sMmTX1. Insets show normalized rise and decay traces which reveal no kinetic differences of Ctrl versus sMmTX1. Values are expressed as mean±SEM, p<0.0001 (****). C, sMmTX1 reduces the amplitude of mini inhibitory postsynaptic currents (mIPSCs) in neurons. Recordings were obtained in the presence of 1 μM TTX, 10 μM DNQX (AMPA and Kainate receptor antagonist) and 40 μM APV (NMDA antagonist) to isolate mIPSCs. Events were recorded for 3 minutes in extracellular solution and then treated for 3 minutes with 300 nM muscimol and 100 nM sMmTX1 before being recorded for 3 more minutes (n=3). Figure shows representative mIPSCs recordings under control and toxin treated conditions showing a clear decrease in the amplitude of the events. D, Quantification of the amplitude decrease is also shown. Data are presented as mean±SEM. (t-test: p<0.0001 for muscimol+sMmTX1 application (****).

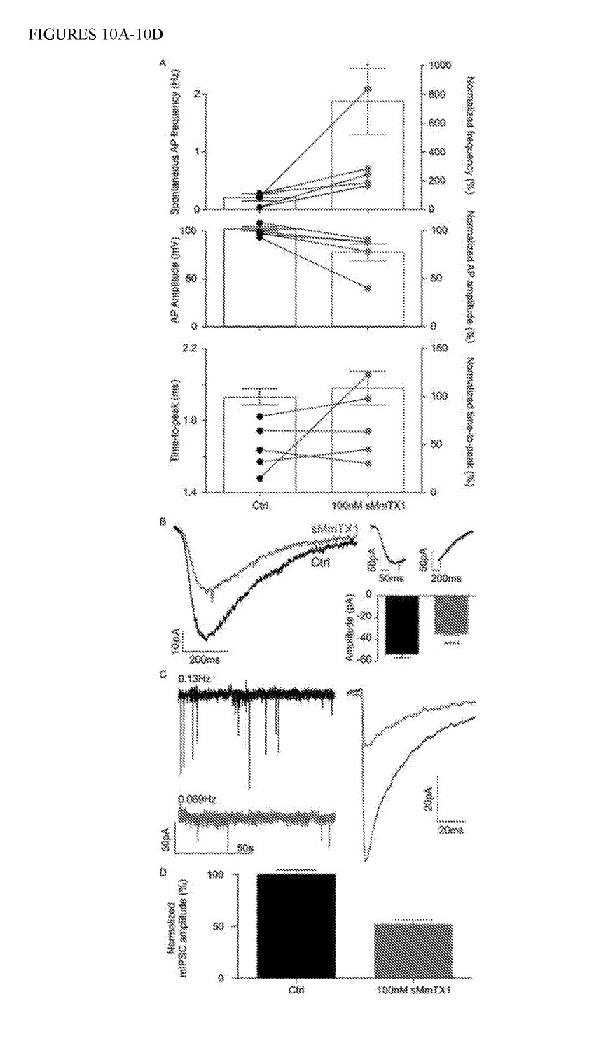

FIG. 11 is an illustration of the sMmTX1 mutations for fluorophore labeling. sMmTX1 was mutated at ⁴His, ²⁸Arg, and ⁴⁷Lys, as well as acetylated at the N-terminus. The two Lys residues of sMmTX1 are mutated into ⁴His, ²⁸Arg and ⁴⁷Ala is mutated into ⁴⁷Lys in order to add a single primary amine, which reacts with the Alexa488 TFP (tetrafluorophenyl) ester, an improvement over the succinimidyl ester (SE or NHS-ester) chemistry typically used to attach fluorophores to the primary amines of biomolecules (the acetylated N-terminus prevents reaction with the primary amine at the N-terminus) (SEQ ID NO: 21).

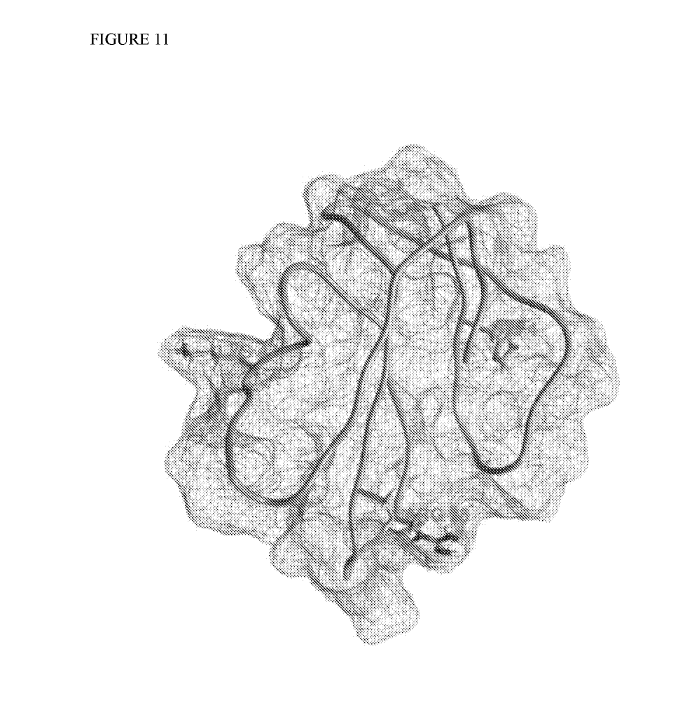

FIG. 12 is a C18 HPLC chromatogram indicating the separation of the unlabeled vs Alexa488-labeled sMmTX1-HRK (SEQ ID NO: 21) toxin.

FIG. 13 depicts the effect of the sMmTX1-HRK mutant, sMmTX1-HRK-conjugated to Alexa488, and sMmTX1-HRK unlabeled HPLC fraction on α1β2γ2 GABA_{A} receptor currents. (Top) A muscimol concentration of 100 nM activates α1β2γ2 GABA_{A} receptors expressed in Xenopus oocytes held at a membrane voltage of −70 mV. When co-applying 100 nM sMmTX1-HRK, currents are potentiated and receptor desensitization tends to accelerate (red trace). Toxin washout is shown on the right (in green). In the presence of 300 nM muscimol (trace below), toxin potentiating effect is diminished whereas desensitization effect is unaltered. (Middle) Expression of the α1β2γ2 receptor in Xenopus oocytes held at a membrane voltage of −70 mV. Protocol consists of two 30-s test applications of 100 nM muscimol separated by a 3-min washout period, followed by 100 nM sMmTX1-HRK-conjugated to Alexa488 application together with 100 nM muscimol. Effect of the conjugated toxin on the receptor is reduced but toxin still binds to the receptor (see FIG. 14). (Bottom) Same protocol as above is used but with sMmTX1-HRK unlabeled HPLC fraction on α1β2γ2 GABA_{A} receptor currents (see FIG. 12). A representative example of four recordings is shown for all traces.

FIG. 14 depicts sMmTX1-HRK conjugated to Alexa488 binding to GABA_{A} receptors in hippocampal cells. sMmTX1-HRK conjugated to Alexa488 was incubated for 45 minutes in culture media together with mouse hippocampal cells (E12 DIV—immature cells). The top figure shows neurons under white light whereas the bottom figure shows the same picture under blue light, thereby exciting the Alexa488 fluorophore. Toxin binding to GABA_{A} receptors can be clearly seen as puncta along immature dendrites.

### DETAILED DESCRIPTION OF THE INVENTION

While surveying the venom of Costa Rican coral snakes, the present inventors identified a major venom fraction that displayed evidence of GABA_{A}-related toxicity in mice. Within this fraction, the polypeptides micrurotoxin1 (MmTX1) and micrurotoxin2 (MmTX2) were identified. The two equally potent peptides have a primary sequence belonging to the PATE-SLURP1-LYNX1-Ly-6/neurotoxin-like family. Extensive binding and competition studies reveal that GABA_{A} receptors are their primary target whereas nAChRs are unaffected. In contrast to PTX which blocks the pore at micromolar concentrations, the data show that MmTX1 and MmTX2 and their variants and derivatives modulate GABA_{A} receptor function at nanomolar quantities by binding to the α+/β− subunit interface, a novel drug binding site with promising therapeutic potential. Electrophysiological experiments with recombinantly and synthetically produced MmTX1 and MmTX2 on hippocampal neurons, HEK 293 cells and Xenopus oocytes expressing common receptor compositions, suggest that these toxins modulate GABA_{A} receptor opening as well as desensitization when co-applied with low concentrations of the agonist muscimol. The inventive peptides demonstrate for the first time that potent and selective GABA_{A}-receptor modulating toxins are present in snake venom and reveal the exciting prospect of discovering novel tools to study these receptors.

In accordance with an embodiment, the present invention provides polypeptides having GABA_{A} modulating activity comprising the following amino acid sequence a) LTCKTCPFTTCPNSESCPGGQSICYQRKWEEHRGERIERRCVANCPAFGSHDTSLLCC TRDNCN (SEQ ID NO: 1), b) a functional fragment of a); c) a functional homolog of a) or b) or functional fragment thereof; and d) a fusion polypeptide comprising an amino acid sequence of any of a) to c).

In accordance with an embodiment, the present invention provides polypeptides having GABA_{A} modulating activity comprising the following amino acid sequence a) LTCKTCPFTTCPNSESCPGGQSICYQRKWEEHHGERIERRCVANCPAFGSHDTSLLCC TRDNCN (SEQ ID NO: 2), b) a functional fragment of a); c) a functional homolog of a) or b) or functional fragment thereof; and d) a fusion polypeptide comprising an amino acid sequence of any of a) to c).

In yet another embodiment, the present invention provides polypeptides having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His Arg Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 6), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Xaa Xaa Cys Lys Thr Cys Pro Phe Thr Thr Cys Pro Asn Ser Glu Ser Cys Xaa Xaa Xaa Xaa Xaa Xaa Cys Tyr Gln Arg Lys Trp Glu Glu His His Gly Glu Arg Ile Glu Arg Arg Cys Xaa Xaa Xaa Cys Pro Ala Phe Gly Ser His Asp Thr Ser Xaa Xaa Cys Cys Thr Arg Asp Asn Cys Asn (SEQ ID NO: 7), b) a functional fragment of a); c) a functional homolog of a) or b) or functional fragment thereof; and d) a fusion polypeptide comprising an amino acid sequence of any of a) to c).

In accordance with a further embodiment, the present invention provides polypeptides having GABA_{A} modulating activity comprising the following amino acid sequence: a) Leu Thr Cys Lys Thr Cys Pro Phe Thr Thr Cys Pro Asn Ser Glu Ser Cys Pro Gly Gly Gin Ser Ile Cys Tyr Gin Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 8), b) a functional fragment of a); c) a functional homolog of a) or b) or functional fragment thereof; and d) a fusion polypeptide comprising an amino acid sequence of any of a) to c).

In accordance with still another embodiment, the present invention provides polypeptides having GABA_{A} modulating activity comprising the following amino acid sequence: a) Met Lys Cys Leu Ile Cys Pro Phe Thr Thr Cys Ser Gin Ser Glu Ser Cys Pro Gly Gly Gin Ser Ile Cys Phe Gln Arg Lys Phe Asp Asp Arg His Gly Asp Arg Ile Glu Arg Gly Cys Ala Val Thr Cys Pro Pro Phe Gly Ser His Asp Thr Ile Phe Cys Cys Ser Thr Asn Asp Cys Asn (SEQ ID NO: 9), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Ile Glu Cys His Asn Cys Pro Phe Thr Thr Cys Gly Asn Ser Glu Ser Cys Pro Gly Gly Gin Ser Ile Cys Val Gin Arg Lys Leu Glu Glu Lys Lys Gly Glu Arg Ile Glu Arg Ser Cys Thr Asp Gly Cys Pro Gly Phe Gly Ser His Asp Thr Val Glu Cys Cys Arg Ile Ala Arg Cys Asn (SEQ ID NO: 10), b) a functional fragment of a); c) a functional homolog of a) or b) or functional fragment thereof; and d) a fusion polypeptide comprising an amino acid sequence of any of a) to c).

In accordance with yet another embodiment, the present invention provides polypeptides having GABA_{A} modulating activity comprising the following amino acid sequence: a) Arg Gin Cys Tyr Thr Cys Pro Phe Thr Thr Cys His Asn Ser Glu Ser Cys Pro Gly Gly Gin Ser Ile Cys Tyr Gin Arg Lys Tyr Glu Glu His Arg Gly Glu Arg Ile Glu Arg Lys Cys Ser Leu Ser Cys Pro Ser Phe Gly Ser His Asp Thr Leu Leu Cys Cys Ala Arg Pro Lys Cys Asn (SEQ ID NO: 11), b) a functional fragment of a); c) a functional homolog of a) or b) or functional fragment thereof; and d) a fusion polypeptide comprising an amino acid sequence of any of a) to c).

In accordance with a further embodiment, the present invention provides polypeptides having GABA_{A} modulating activity comprising the following amino acid sequence: a) Phe Arg Cys Phe Arg Cys Pro Phe Thr Thr Cys Asn Asn Ser Glu Ser Cys Pro Gly Gly Gin Ser Ile Cys Tyr Gin Arg Lys Trp Glu Glu His Arg Gly Glu Arg Ile Glu Arg Arg Cys Val Ala Asn Cys Pro Ala Phe Gly Ser His Asp Thr Leu Leu Cys Cys Lys Arg Glu Glu Cys Asn (SEQ ID NO: 12), b) a functional fragment of a); c) a functional homolog of a) or b) or functional fragment thereof; and d) a fusion polypeptide comprising an amino acid sequence of any of a) to c).

In accordance with still another embodiment, the present invention provides polypeptides having GABA_{A} modulating activity comprising the following amino acid sequence: a) Leu Ser Cys Asn Thr Cys Pro Phe Thr Thr Cys Gln Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 13), b) a functional fragment of a); c) a functional homolog of a) or b) or functional fragment thereof; and d) a fusion polypeptide comprising an amino acid sequence of any of a) to c).

In accordance with another embodiment, the present invention provides polypeptides having GABA_{A} modulating activity comprising the following amino acid sequence: a) Leu Leu Cys Lys Thr Cys Pro Phe Thr Thr Cys Pro Asn Ser Glu Ser Cys Pro Gly Gly Gln Ser Ile Cys Tyr Gln Arg Lys Trp Glu Glu His Arg Gly Glu Arg Ile Glu Arg Arg Cys Val Ala Asn Cys Pro Ala Phe Gly Ser His Asp Thr Leu Leu Cys Cys Thr Arg Asp Asn Cys Asn (SEQ ID NO: 14), b) a functional fragment of a); c) a functional homolog of a) or b) or functional fragment thereof; and d) a fusion polypeptide comprising an amino acid sequence of any of a) to c).

As used herein, the term “Xaa” is a generic descriptor which means any amino acid.

The term, “amino acid” includes the residues of the natural α-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as β-amino acids, synthetic and non-natural amino acids. Many types of amino acid residues are useful in the polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

Reference herein to “derivatives” includes parts, fragments and portions of the inventive GABAergic peptides. A derivative also includes a single or multiple amino acid substitution, deletion and/or addition. Homologues include functionally, structurally or sterochemically similar peptides from venom from the same species of snake or from within the same genus or family of snake. All such homologues are contemplated by the present invention.

Analogs and mimetics include molecules which include molecules which contain non-naturally occurring amino acids or which do not contain amino acids but nevertheless behave functionally the same as the peptide. Natural product screening is one useful strategy for identifying analogs and mimetics.

Examples of incorporating non-natural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A partial list of known non-natural amino acid contemplated herein is shown in Table 1.

TABLE 1
Non-natural Amino Acids

| Non-conventional | | Non-conventional | |
|---|---|---|---|
| amino acid | Code | amino acid | Code |
| α-aminobutyric acid | Abu | L-N-methylalanine | Nmala |
| α-amino-a-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbomyl- | Norb | L-N-methylglutamine | Nmgln |
| carboxylate | | L-N-methylglutamic acid | Nmglu |
| cyclohexylalanine | | Chexa L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methyloniithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Analogs of the subject peptides contemplated herein include modifications to side chains, incorporation of non-natural amino acids and/or their derivatives during peptide synthesis and the use of crosslinkers and other methods which impose conformational constraints on the peptide molecule or their analogs.

In accordance with an embodiment, the present invention provides a sMmTx1-HRK mutant polypeptide having GABA_{A} modulating activity comprising the following amino acid sequence with an acetyl functional group at the N-terminus ((Acl) a) (Acl)LTCHTCPFTTCPNSESCPGGQSICYQRRWEEHRGERIERRCVANCPKFGSHDTSL LCCTRDNCN (SEQ ID NO: 21), b) a functional fragment of a); c) a functional homolog of a) or b) or functional fragment thereof, and d) a fusion polypeptide comprising an amino acid sequence of any of a) to c).

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

The present invention further contemplates small chemical analogs of the subject peptides capable of acting as antagonists or agonists of the GABAergic peptides of the present invention. Chemical analogs may not necessarily be derived from the peptides themselves but may share certain conformational similarities. Alternatively, chemical analogs may be specifically designed to mimic certain physiochemical properties of the peptides. Chemical analogs may be chemically synthesized or may be detected following, for example, natural product screening.

The term, “peptide,” as used herein, includes a sequence of from four to 100 amino acid residues in length, preferably about 10 to 80 residues in length, more preferably, 15 to 65 residues in length, and in which the α-carboxyl group of one amino acid is joined by an amide bond to the main chain (α- or β-) amino group of the adjacent amino acid. The peptides provided herein for use in the described and claimed methods and compositions can also be cyclic.

The precise effective amount for a human subject will depend upon the severity of the subject's disease state, general health, age, weight, gender, diet, time and frequency of administration, drug combination(s), reaction sensitivities, and tolerance or response to therapy. A routine experimentation can determine this amount and is within the judgment of the medical professional. Compositions may be administered individually to a patient, or they may be administered in combination with other drugs, hormones, agents, and the like.

Routes of administration of the inventive peptides include, but are not limited to intravenously, intraperitioneal, subcutaneously, intracranial, intradermal, intramuscular, intraocular, intrathecal, intracerebrally, intranasally, infusion, orally, rectally, via iv drip, patch and implant.

In one or more embodiments, the present invention provides pharmaceutical compositions comprising one or more of the inventive peptides and a pharmaceutically acceptable carrier. In other aspects, the pharmaceutical compositions also include one or more additional biologically active agents.

With respect to peptide compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In addition, in an embodiment, the compositions comprising the inventive peptides or derivatives thereof, may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular peptide containing compositions, as well as by the particular method used to administer the composition. Accordingly, there are a variety of suitable formulations of the pharmaceutical compositions of the invention. More than one route can be used to administer the compositions of the present invention, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

In accordance with an embodiment, the present invention provides a composition comprising one or more polypeptides having GABA_{A} modulating activity described herein, and at least one or more biologically active agent.

As used herein the term “therapeutically active agent” or “biologically active agent” means an agent useful for the treatment or modulation of a disease or condition in a subject suffering therefrom. Examples of therapeutically active agents can include any drugs, peptides, siRNAs, and conjugates, known in the art for treatment of disease indications.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Further examples of biologically active agents include, without limitation, enzymes, receptor antagonists or agonists, hormones and antibodies. Specific examples of useful biologically active agents include, for example, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, and adrenergic agonist sympathomimetics intravenous anesthetics, barbiturate intravenous anesthetics, benzodiazepine intravenous anesthetics, and opiate agonist intravenous anesthetics skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, such as antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, and hypnotics, barbiturate sedatives and hypnotics, benzodiazepine anxiolytics, sedatives, and hypnotics, and psychostimulants.

Therefore, in accordance with an embodiment, the present invention provides the use of the inventive polypeptides disclosed herein, for modulating GABA_{A} receptors in a cell or population of cells expressing the GABA_{A} receptor comprising contacting the cell or population of cells with an effective amount of the inventive polypeptides.

In accordance with another embodiment, the present invention provides the use of the inventive polypeptides disclosed herein, for modulating GABA_{A} receptors in a subject suffering from a neurological disorder, comprising administering to the subject, an effective amount of a composition comprising one or more polypeptides disclosed herein.

In accordance with a further embodiment, the present invention provides the use of the inventive polypeptides disclosed herein, for modulating GABA_{A} receptors in a subject suffering from a neurological disorder, comprising administering to the subject, an effective amount of a composition comprising one or more polypeptides disclosed herein, and at least one or more biologically active agents.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect.

As used herein, the term “treat,” as well as words stemming therefrom, includes diagnostic and preventative as well as disorder remitative treatment.

In accordance with an embodiment, the present invention provides methods for modulating GABA_{A} receptors in a subject suffering from a neurological disorder comprising administering to the subject a composition comprising one or more polypeptides having GABA_{A} modulating activity described herein, and at least one or more biologically active agents.

Neurological disorders which involve, either directly or indirectly, GABAergic function may be studied and/or treated using the peptides and pharmaceutical compositions comprising the inventive peptides. Examples of such diseases include, but are not limited to, affective disorders such as depression, bipolar disorder, generalized anxiety disorders, epilepsy, convulsant disorders, schizophrenia, and certain pain disorders, Stiff-person syndrome (SPS), pathophysiology of the fragile X syndrome, tonic inhibition neuronal dysfunctions, Down syndrome, autism, and generalized seizures.

In accordance with an embodiment, the present invention provides methods for modulating GABA_{A} receptors in a cell or population of cells expressing the GABA_{A} receptor comprising contacting the cell or population of cells with an effective amount of the inventive polypeptides described herein. It is contemplated that the inventive GABA_{A} modulating peptides and compounds will be useful in studying neurological functions in vitro and in vivo.

In some embodiments, the inventive peptides and compositions can include imaging agents covalently linked to the peptides and compositions.

In accordance with an embodiment, the present invention provides a composition comprising one or more polypeptides having GABA_{A} modulating activity described herein, and at least one or more imaging agents.

In some embodiments, the imaging agent is a fluorescent dye. The dye may be an emitter in the visible or near-infrared (NIR) spectrum. Known dyes useful in the present invention include carbocyanine, indocarbocyanine, oxacarbocyanine, thuiicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor488, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4α-diaza-s-indacene) and sharp fluorescence with high quantum yield and excellent thermal and photochemical stability.

Other imaging agents which can be attached to the inventive peptides and compositions of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F. In accordance with an embodiment, the chelating agent is DOTA.

In accordance with an embodiment, the present invention provides a nanoplex molecule wherein the reporter portion comprises a metal isotope suitable for imaging. Examples of isotopes useful in the present invention include Tc-94m, Tc-99m, In-111, Ga-67, Ga-68, Y-86, Y-90, Lu-177, Re-186, Re-188, Cu-64, Cu-67, Co-55, Co-57, Sc-47, Ac-225, Bi-213, Bi-212, Pb-212, Sm-153, Ho-166, or Dy-i66.

In accordance with an embodiment, the present invention provides peptides and compositions wherein the imaging agent portion comprises ¹¹¹In labeled DOTA which is known to be suitable for use in SPECT imaging.

In accordance with another embodiment, the present invention provides a peptides and compositions wherein the imaging agent comprises Gd³⁺ labeled DOTA which is known to be suitable for use in MR imaging. It is understood by those of ordinary skill in the art that other suitable radioisotopes can be substituted for ¹¹¹In and Gd³⁺ disclosed herein.

In accordance with an embodiment, the present invention provides one or more nucleic acid sequences encoding any of the polypeptides having GABA_{A} modulating activity or derivatives, homologues, analogues or mimetics thereof disclosed herein.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered intemucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

In accordance with an embodiment, the present invention provides one or more non-naturally occurring cDNA sequences encoding any of the polypeptides having GABA_{A} modulating activity or derivatives, homologues, analogues or mimetics thereof disclosed herein.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acids can be constructed based on chemical synthesis and/or enzymatic ligation reactions using procedures known in the art. See, for example, Sambrook et al. (eds.), Molecular Cloning, A Laboratory Manual, 3rd Edition, Cold Spring Harbor Laboratory Press, New York (2001) and Ausubel et al., Current Protocols in Molecular Biology, Greene Publishing Associates and John Wiley & Sons, NY (2007). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

In accordance with another embodiment, the present invention provides a vector comprising one or more nucleic acid sequences encoding any of the polypeptides having GABA_{A} modulating activity or derivatives, homologues, analogues or mimetics thereof disclosed herein.

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring intemucleotide linkages, or both types of linkages. Preferably, the non-naturally occurring or altered nucleotides or intemucleotide linkages do not hinder the transcription or replication of the vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell, such as Xenopus oocytes. Replication systems can be derived, e.g., from ColE1, 2μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA or RNA based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, LacZ, green fluorescent protein (GFP), luciferase, neomycin/G418 resistance genes, hygromycin resistance genes, histidinol resistance genes, tetracycline resistance genes, and ampicillin resistance genes.

The heterologous nucleic acid can be a nucleic acid not normally found in the target cell, or it can be an extra copy or copies of a nucleic acid normally found in the target cell. The terms “exogenous” and “heterologous” are used herein interchangeably.

The invention further provides a host cell comprising any of the recombinant expression vectors described herein. As used herein, the term “host cell” refers to any type of cell that can contain the inventive recombinant expression vector. The host cell can be an animal cell. Preferably, in an embodiment, the host cell is a mammalian cell. The host cell can be a cultured cell or a primary cell, i.e., isolated directly from an organism, e.g., a human. The host cell can be an adherent cell or a suspended cell, i.e., a cell that grows in suspension. Most preferably, the host cell is a human cell. The host cell can be of any cell type, can originate from any type of tissue, and can be of any developmental stage. Most preferably the host cells can include, for instance, muscle, lung, and brain cells, and the like.

The host referred to in the inventive methods can be any host. Preferably, the host is a mammal.

As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a lung cell), which does not comprise any of the recombinant expression vectors, or a cell other than a lung cell, e.g., a skin cell, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

### EXAMPLES

Purification and Production of MmTX1 and MmTX2.

MmTX1 and MmTX2 were purified from Micrurus mipartitus venom from Costa Rica by a two-step HPLC protocol. Edman sequencing was carried out on a cysteine reduced and s-carboxymethylated derivative. Recombinant expression of MmTX1/2 was based on a synthetic gene and site-directed mutagenesis was executed with the quick-change mutagenesis kit (Stratagene). All constructs were sequenced (Genome express) and expressed in E. Coli HB 101 (Promega) as a fusion protein with the ZZ domain (GE Healthcare). Fusion proteins were purified on an IgG-Sepharose column (GE Healthcare) and toxin-moiety kept free from the ZZ domain using cyanogen bromide. Except where indicated, all chemicals were purchased from Sigma-Aldrich. sMmTX1 was chemically synthesized using standard solid phase peptide synthesis methodology and Fmoc chemistry. The 5-disulfide bridges were allowed to fold in a 100 mM Tris-HCl buffer, 1 mM EDTA, pH 9.0, adding a combination of GSSG/GSH in a 1 mM/2 mM ratio and sMmTX1 at a concentration of 20 μM. Purification and desalting was achieved by HPLC.

Template-Based 3D Modelling.

Homology modelling of MmTX1 was performed with MODELLER using ModWeb Server version SNV.r1368M within ModBase. Visualisation and drawing of selected models were accomplished with UCSF Chimera.

MmTX2 Iodination and Pharmacological Experiments.

Na¹²⁵I and [³H]muscimol were obtained from PerkinElmer and isoguvacine from Peninsula laboratories. All other reagents were from Sigma-Aldrich. MmTX2 was radioactively labeled by lacto-peroxidase-catalyzed iodination with specific activities of 900 Ci/mmol routinely obtained. Synaptosome fractions (P2) of Wistar rat brains were lysed on ice by dilution into 15 volumes of hypotonic buffer, and SPMs were pelleted at 12,500×g, 20 minutes at 5° C. Binding experiments were performed in 1.5 ml tubes in buffer 10 mM Tris, 100 mM NaCl, 1 mg/ml BSA, pH 7.4, 50 μg of SPM protein. The incubations were performed at 37° C. (¹²⁵I-MmTX2) or 4° C. ([³H]muscimol). Data analysis was performed using GraphPad PRISM®. Data are reported as mean±SEM.

Electrophysiological Recordings.

HEK 293 cells transfected with the α1β2γ2 GABA_{A} receptor and GFP were patch-clamped under a water-immersed 40× objective of a Zeiss Axioskop 2 FS Plus. Borosilicate glass capillaries (Harvard Apparatus) were pulled (Sutter Instruments) and had a resistance of 3-5 MΩ when filled with the internal solution. Membrane currents were recorded using Pulse and Patchmaster software (HEKA) in combination with an EPC-9 patch-clamp amplifier (HEKA). Data were low-pass filtered at 2.9 kHz. Only data from recordings with an access resistance <20 MΩ were evaluated. Data were analyzed using Fitmaster (HEKA), Igor Pro 6.03 (Wavemetrics) and Excel (Microsoft). Averaged values are given as mean±SEM.

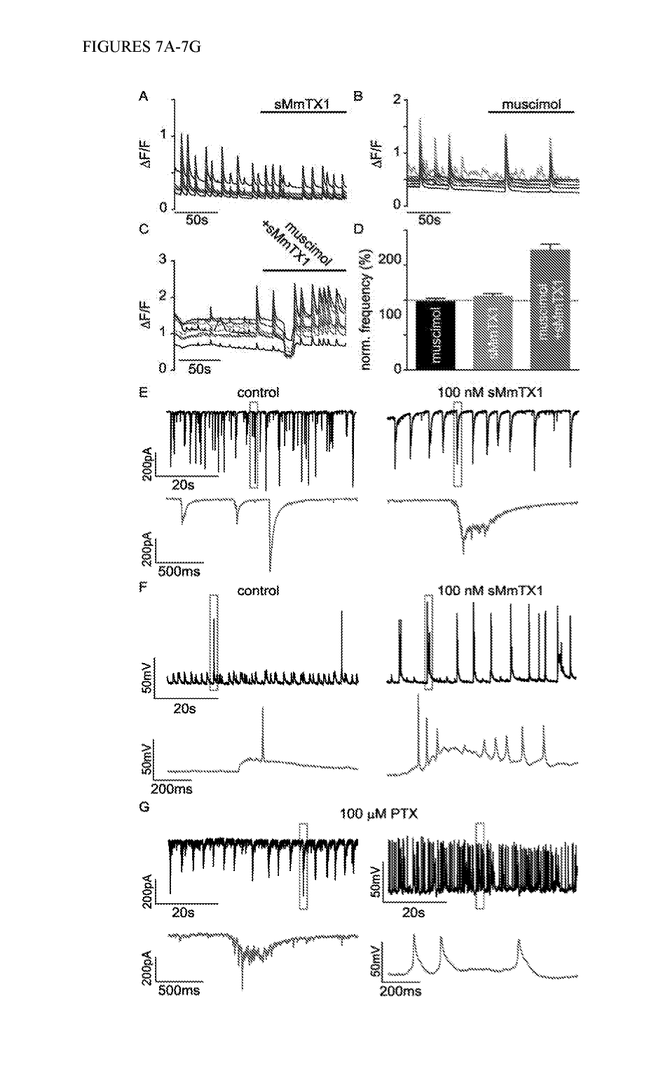

Hippocampal cell cultures were prepared from P1 mice (C57B16J). Whole-cell patch clamp experiments were performed on hippocampal neurons (DIV 14) at room temperature (˜22° C.) and the holding potential was −70 mV. The extracellular solution used in hippocampal neuron experiments contained (in mM): 143 NaCl, 5 KCl, 0.8 MgCl₂, 1 CaCl₂, 10 HEPES, 5 glucose, 0.5 μM TTX, 10 μM CNQX, and 20 μM APV, pH=7.3 with NaOH. In experiments with HEK 293 cells the same extracellular solution was used without TTX, CNQX, and APV. The pipette solution contained (in mM): 140 CsCl, 1 CaCl₂, 1 MgCl₂, 11 EGTA, 5 HEPES; pH adjusted to 7.2 with CsOH. For recordings in FIG. 7, ACSF solution contained (in mM): 140 NaCl, 5 KCl, 2 CaCl₂, 2 MgCl₂, 10 HEPES, and 10 Glucose (pH 7.3 adjusted with NaOH). Recordings were performed with an Axopatch 200B amplifier equipped with Digidata 1440 and pClamp 10 (Molecular Devices). Whole-cell recordings were done using borosilicate glass pipettes (4-6 MΩ) filled with internal solution containing (in mM): 135 K-gluconate, 20 KCl, 10 HEPES, 4 Mg-ATP, 0.3 Na₂GTP, 0.5 EGTA.

Rat GABA_{A} receptor subunits were also expressed in Xenopus oocytes. cRNA was synthesized using T7 polymerase (Life Technologies) after linearizing the DNA with appropriate restriction enzymes. Currents were studied following 1-2 days incubation after cRNA injection using two-electrode voltage-clamp recording techniques (OC-725C, Warner Instruments). Microelectrode resistances were 0.5-1 MΩ when filled with 3 M KCl. The external recording solution contained (in mM) 100 NaCl, 5 HEPES, 1 MgCl₂ and 1.8 CaCl₂, pH=7.6 with NaOH. All experiments were performed at room temperature (˜22° C.). Data analysis was performed using Clampfit10 (Molecular Devices), and Origin 8 (OriginLab).

Calcium Imaging of Hippocampal Neurons.

Hippocampi were obtained from Sprague Dawley rat embryos at embryonic day 18. Cells were plated over coverslips coated with Laminin (Life Technologies) and poly-D-Lysine (Sigma-Aldrich). Astrocyte beds were prepared at a density of 80,000 cells/ml and cultured in DMEM (Life Technologies) with 10% Fetal Bovine Serum, 6 mM glutamine in 5% CO₂ at 37° C. Neurons were plated over 14 days in vitro confluent astrocyte beds at a density of 150,000 cells/ml and cultured for 3 weeks in Neurobasal supplemented with B27 and 2 mM glutamax. Neurons were incubated with 2 m Fluo-4 AM (Life Technologies) for 15 minutes. The images were obtained with an Olympus BX51WI microscope and a Lambda DG-4 wavelength switcher (Sutter Instruments) at 2 Hz. The recording chamber was perfused at 2 ml/min at 32° C. with ACSF. Calcium spikes were analyzed with MiniAnalysis (Synaptosoft). Statistical analysis was performed using GraphPad PRISM®. Data was analyzed with paired Student's test.

### Example 1

Identification of MmTX1 and MmTX2 in Coral Snake Venom.

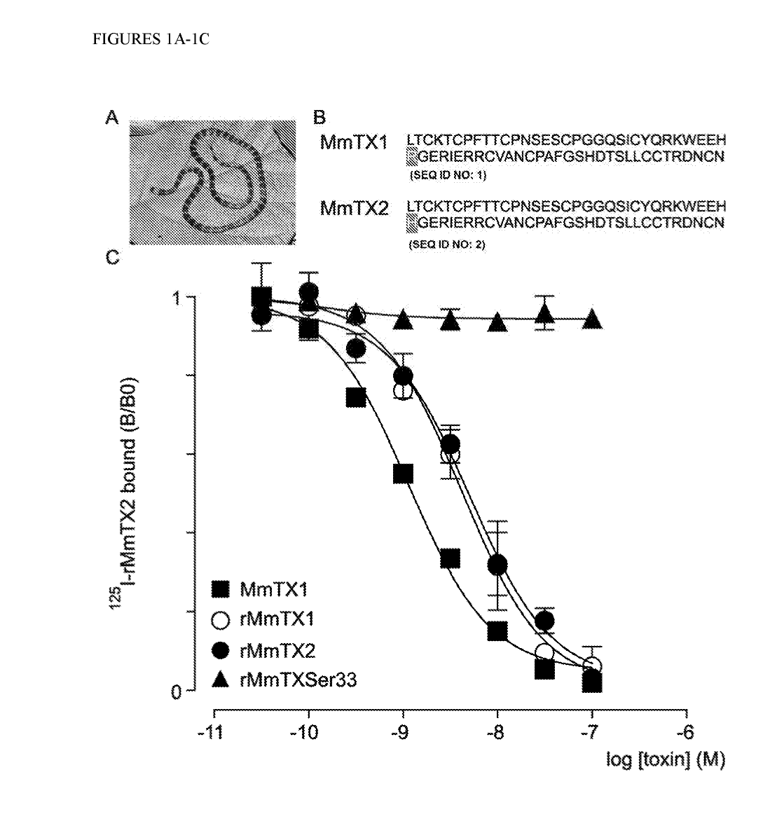

When using competitive binding assays on the Torpedo electric organ to search for novel α-neurotoxins active on nAChRs, we identified a major but inactive venom fraction from the Costa Rican coral snake Micrurus mipartitus. However, intracerebroventricular injection of this fraction into mice (LD₅₀=0.002 mg/kg) resulted in periods of reduced basal activity followed by bursts of intense seizures. Intrigued by this observation, we purified this fraction and identified two components (data not shown). Automated Edman sequencing combined with mass spectrometry revealed two amino acid sequences of 64 residues containing 10 cysteine residues each (FIG. 1A). Both peptides differ in one amino acid at position 33 with either an arginine (MmTX1, 7205.0 Da) or a histidine (MmTX2, 7186.0 Da).

A BLAST search revealed that MmTX1 and MmTX2 are novel members of the PATE-SLURP 1-LYNX1-Ly-6/neurotoxin-like family. When combining the BLAST output with PHYLIP, we find a phylogeny pattern that classifies all homologous toxins into five clades (data not shown). Remarkably, the first clade consists exclusively of toxins found in Micrurus species, with MmTX1 and MmTX2 having the highest sequence identity. The availability of numerous three-finger snake toxin structures allowed us to reliably model MmTX1 using MODELLER. The program selected γ-bungarotoxin (PDB: 1MR6) and candoxin (PDB: 1JGK) as templates with a good ModPipe quality score of 1.5674 (data not shown). As expected, the predicted tertiary structure of MmTX1 fits within the three-fingered snake toxin family with one of the five disulfide bridges located within the first loop-finger. As such, MmTX1 can be classified as a member of the Elapid weak-toxin subgroup of which the biological activity has not yet been defined. Therefore, it is possible that other toxins within this subgroup interact with the same molecular target as MmTX1. When comparing MmTX1 to α-neurotoxins that specifically target nAChRs, substantial amino acid differences within the first loop are evident. However, the polar arginine (MmTX1) or histidine (MmTX2) at position 33 at the apex of the second loop is conserved. As can be seen in the crystal structure of α-cobratoxin in complex with AChBP, a structural and functional surrogate of the nAChR ligand-binding domain, the polar residue in this location comes in close contact with the receptor and may be vital for toxin activity.

### Example 2

Production of MmTX1 and MmTX2.

Micrurus mipartitus is a rare Costa Rican snake species which delivers small quantities of venom. Therefore, we developed a periplasmic expression system with the goal of producing large amounts of recombinant (r)MmTX1 and rMmTX2. The in vivo activity of rMmTX1 and rMmTX2 was assayed using microinjections into mouse brain and resulted in a median lethal dose (LD₅₀) of 0.027 mg/kg and 0.010 mg/kg, respectively (n=9), a result that corresponds to that of the native fraction (LD₅₀=0.002 mg/kg). Moreover, similar behavioral signs including reduced activity combined with severe seizures were noted. In contrast, rMmTX1 and rMmTX2 are inactive when injected intravenously into mice at doses up to 1 mg/kg. This phenotype is markedly different from that observed when assaying α-neurotoxins which target nAChRs to produce a flaccid paralysis. As such, these results suggest that MmTX1 and MmTX2 may target GABA_{A} receptors expressed in the CNS.

In addition to establishing a recombinant expression system, we synthesized (s)MmTX1 using a Fmoc strategy. Correct folding of the five disulfide bridges was achieved by using controlled redox buffering (see above: Purification and production of MmTX1 and MmTX2). To verify proper toxin function, we injected sMmTX1 into mouse brain and found: 1) an LD₅₀ value similar to that of rMmTX1 (0.013 mg/kg); and 2) an identical mouse behavioral phenotype. These results indicate that native, recombinant, and synthetic MmTX1/2 are equally effective in influencing their target. Next, we tested whether these toxins bind to receptors expressed in rat brain synaptic plasma membranes (SPMs).

### Example 3

Binding of rMmTX1 and rMmTX2 to Rat Brain Synaptic Plasma Membranes.

To assess the activity of rMmTX1 and rMmTX2 in SPMs, we performed competitive binding experiments between ¹²⁵I-rMmTX2 and wild-type (WT) MmTx2, rMmTx1, and rMmTx2 (FIG. 1). The equilibrium inhibition constant (Kᵢ) of WT MmTX2 (Kᵢ=0.8 nM; log K=−9.09±0.07) is comparable to that found for rMmTX1 (Kᵢ=3.0 nM; log Kᵢ=−8.52±0.10) and rMmTX2 (Kᵢ=3.7 nM; log Kᵢ=−8.44±0.11). Moreover, no difference in Kᵢ is observed between rMmTX1 and rMmTX2. Since the arginine found in the tip of the central loop helps determine three-finger α-neurotoxin toxin activity towards nAChRs, we examined whether this amino acid position also contributes to the biological activity of rMmTx2. To this end, we mutated the endogenous histidine to the smaller polar residue serine (rMmTx2Ser33) and measured competitive binding with ¹²⁵I-rMmTX2. As a result, we found that this mutation ablates rMmTx2 function suggesting that the amino acid at this position is indeed vital for rMmTx2 activity (FIG. 1B).

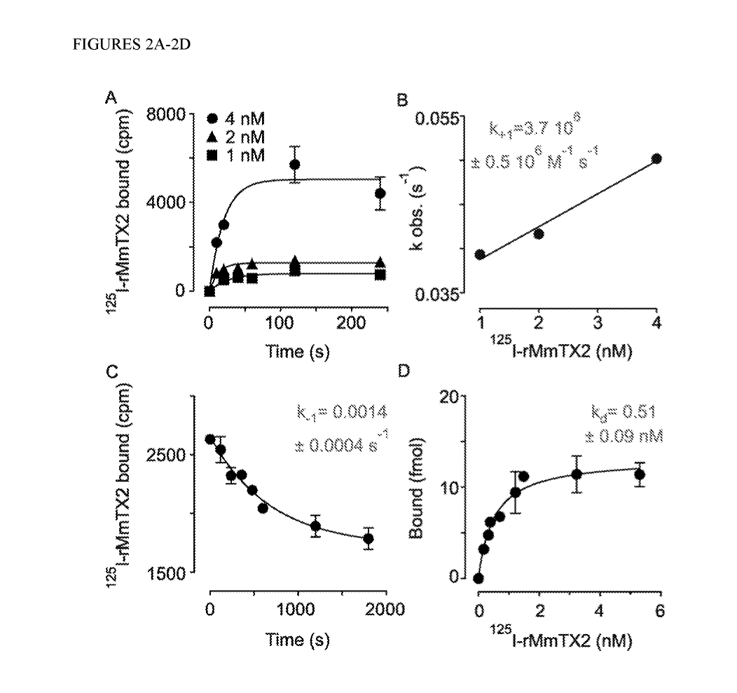

Next, we investigated the binding rate of ¹²⁵I-rMmTX2 to SPMs using concentrations ranging from 1 to 4 nM (FIG. 2A) and fitting the data by a single exponential association curve. Plotting each observed kinetic constant against the respective ¹²⁵I-rMmTx2 concentration yields a second-order kinetic constant of association (k+1) of 3.7±0.5×10⁶ M⁻¹ s⁻¹ (FIG. 2B). The dissociation process of ¹²⁵I-rMmTX2 bound to SPMs was determined after the addition of a 1000-fold excess of unlabeled rMmTX2 (FIG. 2C). Fitting the data with a single exponential decay curve results in a first-order kinetic constant (k−1) of 0.0014±0.0004 s⁻¹ and a half time duration of ˜8 minutes. From the ratio of both kinetic constants, we can deduce a true dissociation value (K_{d}=k−1/k+1) of 0.38±0.05 nM. Finally, we conducted a SPM saturation binding experiment at equilibrium using ¹²⁵I-rMmTX2 concentrations up to 6 nM. Non-specific binding was determined in the presence of a 1000-fold excess of unlabeled rMmTX2 and subtracted to obtain specific binding data (FIG. 2D). The results of this experiment indicate the presence of a single class of non-interacting binding sites with a K_{d} of 0.51±0.09 nM and a maximum capacity Bₘₐₓ of 264±14 fmol/mg of synaptic protein.

### Example 4

Binding of rMmTX2 to GABA_{A} Receptors in SPMs.

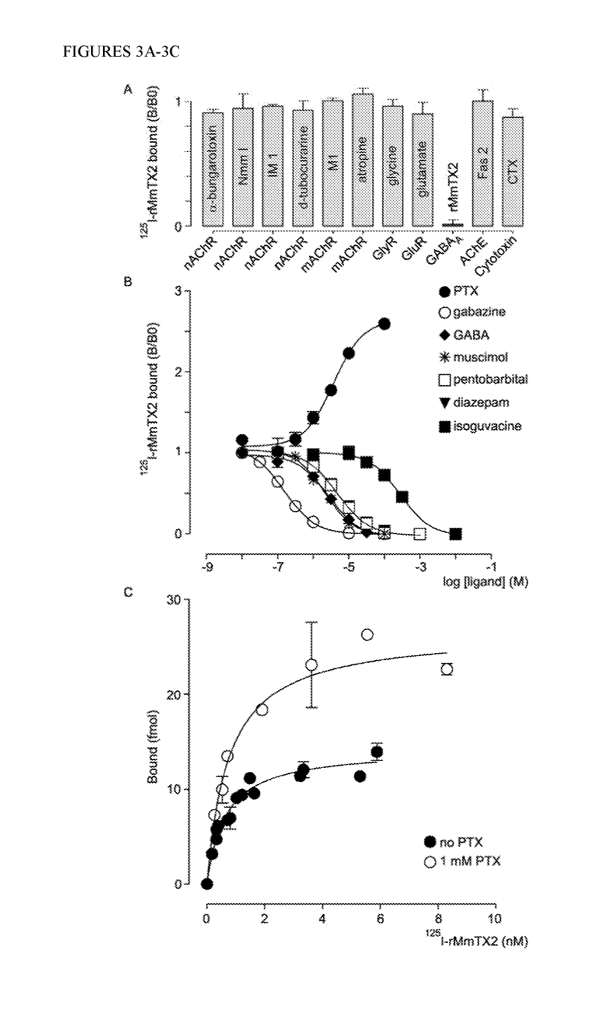

To identify the primary target of rMmTx2 in SPMs, we carried out competitive binding experiments with a subset of non-peptidic and peptidic modulators of nAChRs, muscarinic (m)AChRs, GABA_{A} receptors, GlyRs, glutamate receptors (GluRs), and acetylcholinesterase (AChE). Given its close structural relation to members of the three-finger α-neurotoxin family that bind to nAChRs, we initially investigated whether rMmTx2 competes with short- and long-chain snake α-neurotoxins (Nmm I and α-bungarotoxin) as well as marine snail α-conotoxins (M1 and IM 1) and a plant toxin (d-tubocurarine) that target this particular receptor (FIG. 3A). Strikingly, no competition is observed suggesting that nAChRs are not the primary target of rMmTX2. In concert, we observe no flaccid paralysis when injecting rMmTX2 intravenously into mice. Similarly, rMmTX2 does not compete with atropine, glycine, glutamate, and Fas 2 that target mAChRs, GlyRs, GluRs, and AChE, respectively. Finally, rMmTX2 does not displace cardiotoxin (CTX), a cytotoxic membrane-targeting peptide that also belongs to the three-finger α-neurotoxin structural family (FIG. 3A).

We next examined whether ¹²⁵I-MmTX2 targets GABA_{A} receptors in SPMs by conducting competitive binding experiments with receptor agonists, antagonists, as well as allosteric modulators (FIG. 3B). As a result, we found that the competitive GABA_{A} receptor antagonist gabazine is the most effective in reducing ¹²⁵I-MmTX2 binding to SPMs (EC₅₀=0.2 μM; log EC₅₀=−6.81±0.03; nH=1.00±0.07). In addition, the agonists GABA (EC₅₀=2.7 μM; log EC₅₀=−5.56±0.08; nH=0.99±0.18) and muscimol (EC₅₀=2.1 μM; log EC₅₀=−5.67±0.09; nH=1.08±0.24) reduce toxin binding to a similar extent. Moreover, the positive allosteric modulators diazepam (EC₅₀=4.9 μM; log EC₅₀=−5.30±0.04; nH=−0.96±0.07) and pentobarbital (EC₅₀=4.8 μM; log EC₅₀=−5.32±0.03; nH=0.98±0.07) are equally potent in displacing ¹²⁵I-MmTX2. In contrast, the partial agonist isoguvacine (EC₅₀=275 μM; log EC₅₀=−3.56±0.10; nH=1.02±0.29) is the least efficient in reducing ¹²⁵I-MmTX2 binding.

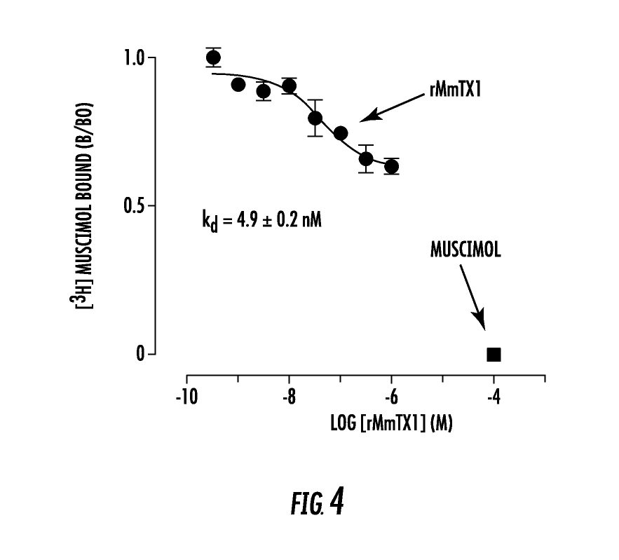

Surprisingly, the non-competitive channel blocker PTX (EC₅₀=3.7 μM; log EC₅₀=−5.43±0.07; nH=+1.05±1.16) markedly potentiates ¹²⁵I-MmTX2 binding capacity to SPMs. To distinguish between an effect of PTX on ¹²⁵I-MmTX2 binding affinity and binding site capacity, we repeated a binding experiment at equilibrium in the presence of a saturating PTX concentration (1 mM) (FIG. 3C). By comparing binding isotherm constants, we find that PTX potentiates ¹²⁵I-MmTX2 binding capacity without effecting toxin affinity (K_{d} with PTX=0.76±0.18 nM, Bₘₐₓ=532±33 fmol/mg of SPM protein giving a ratio with/without PTX of 2.01). Finally, we considered if rMmTX1 has an effect on [³H]muscimol binding to SPMs. Since GABA_{A} receptors possess a high- and low-affinity muscimol binding site, we conducted this experiment at saturating concentrations (40 nM). Using a nonlinear fit for an allosteric modulator, we found that rMmTX1 is able to compete with [³H]muscimol with a K_{d} of 4.9±0.2 nM and only for about 40% of the total [³H]muscimol binding capacity (FIG. 4) suggesting that rMmTX1 may only influence one muscimol binding site. Altogether, our results indicate that rMmTX2, and by extension rMmTX1, preferentially targets GABA_{A} receptors with high affinity. Moreover, a variety of allosteric and orthosteric compounds compete with toxin binding whereas the pore-blocking molecule PTX doubles toxin binding capacity. Next, we explored toxin activity on mouse hippocampal neurons that express GABA_{A} receptors. Since rMmTX1, rMmTX2, and sMmTX1 have comparable effects in our mouse and binding assays, we consider both toxins as well as their synthesis route to be interchangeable.

### Example 5

MmTX1 Influences GABA_{A} Receptor-Mediated Currents in Hippocampal Neurons.

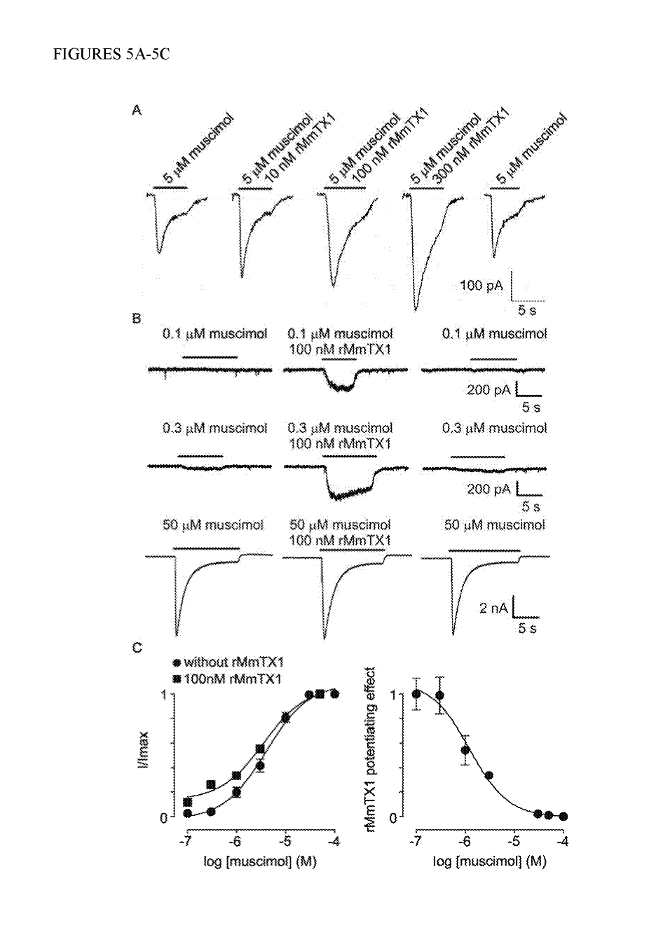

To test the biological activity of rMmTX1 on mouse brain hippocampal neurons, we first applied 100 nM in the absence of an agonist and observed no effect on GABA_{A} receptors. In contrast, we see a potentiating effect of the toxin in the presence of 5 μM muscimol: rMmTX1 increases the amplitude of the GABA_{A} receptor-mediated current elicited by 5 μM muscimol in a dose-dependent manner (FIG. 5, Table 2). In the absence of rMmTX1, 0.1 μM muscimol fails to elicit a detectable current (5 out of 7 neurons). However, we observe a Cl⁻ current of 228±30 pA when 0.1 μM muscimol is applied together with 100 nM rMmTX1. In 3 of 7 neurons, 0.3 μM muscimol induces no response, whereas application of 0.3 μM muscimol together with 100 nM rMmTX1 generates a large inward current (439±51 pA). In the remaining 4 out of 7 neurons, 0.3 μM muscimol induces a small inward current which is strongly potentiated by simultaneous application of 100 nM rMmTX1. In contrast to the potentiating effect of rMmTX1 in the lower range of muscimol concentrations, we do not observe a toxin-induced potentiation at muscimol concentrations greater than 10 μM. Upon determining the concentration-dependence of GABA_{A} receptor currents by muscimol alone, we find an EC₅₀ of 4.1 μM and an nH coefficient of 1.4, which is in good agreement with reported values for GABA_{A} receptors (5.4-24 μM and nH between 1.1 and 1.6). When surveying all muscimol concentrations tested without and in the presence of 100 nM rMmTX1, it is clear that the toxin causes GABA_{A} receptor-mediated currents to increase up to 7-fold at low agonist concentrations whereas saturating muscimol quantities suppress toxin effects.

Table 3 summarizes the factors by which the current amplitudes are potentiated by 100 nM rMmTX1 in dependence of the muscimol concentration. From these data the rMmTX1-induced portion of the current amplitude was determined and plotted versus muscimol concentration which reveals that the half-maximal potentiating effect of rMmTX1 is obtained at about 1 μM muscimol. Altogether, our data suggest that rMmTX1 potentiates GABA_{A} receptor activation in a non-saturating concentration range of muscimol. However, an increase in inward Cl⁻ currents explains the initial rest behavior observed in mice upon toxin injection into the brain whereas the ensuing seizures resemble those observed when inhibiting GABA_{A} receptors with PTX. To further investigate the mechanism underlying MmTX1/2 action, we tested the toxins on common GABA_{A} receptor variants expressed in heterologous systems.

TABLE 2
Potentiation of GABA_{A} receptor currents elicited by 5 μM muscimol
together with increasing rMmTX1 concentrations.

| 5 μM | 5 μM | 5 μM | 5 μM |
|---|---|---|---|
| muscimol + | muscimol + | muscimol + | muscimol + |
| 1 nM rMmTX1 | 10 nM rMmTX1 | 100 nM rMmTX1 | 300 nM rMmTX1 |
| 1.24 ± 0.07 | 1.22 ± 0.03 | 1.99 ± 0.28 | 1.69 ± 0.25 |

The factor by which the amplitudes of the GABA_{A} currents were increased was obtained by dividing the current amplitudes elicited by 5 μM muscimol+rMmTX1 by the control current amplitudes; mean±SEM; n=9. p-values for 1, 10, 100, and 300 nM are 0.0077, 0.00058, 0.011, and 0.027 respectively.

TABLE 3
Potentiation of GABA_{A} receptor currents elicited by increasing
concentrations of muscimol in the presence of 100 nM rMmTX1.

| [muscimol] + | Factor of GABA_{A} current |
|---|---|
| 100 nM rMmTX1 | increase |
| 0.1 μM | 7.9 ± 2.9 (n = 2) |
| 0.3 μM | 7.5 ± 1.2 (n = 4) |
| 1.0 μM | 1.9 ± 0.4 (n = 3) |
| 3 μM | 1.4 ± 0.1 (n = 6) |
| 30 μM | 1.0 ± 0.1 (n = 4) |
| 50 μM | 1.0 ± 0.1 (n = 3) |

Values shown are mean ± SEM.

### Example 6

sMmTX1 Modulates Heterologously Expressed GABA_{A} Receptors.

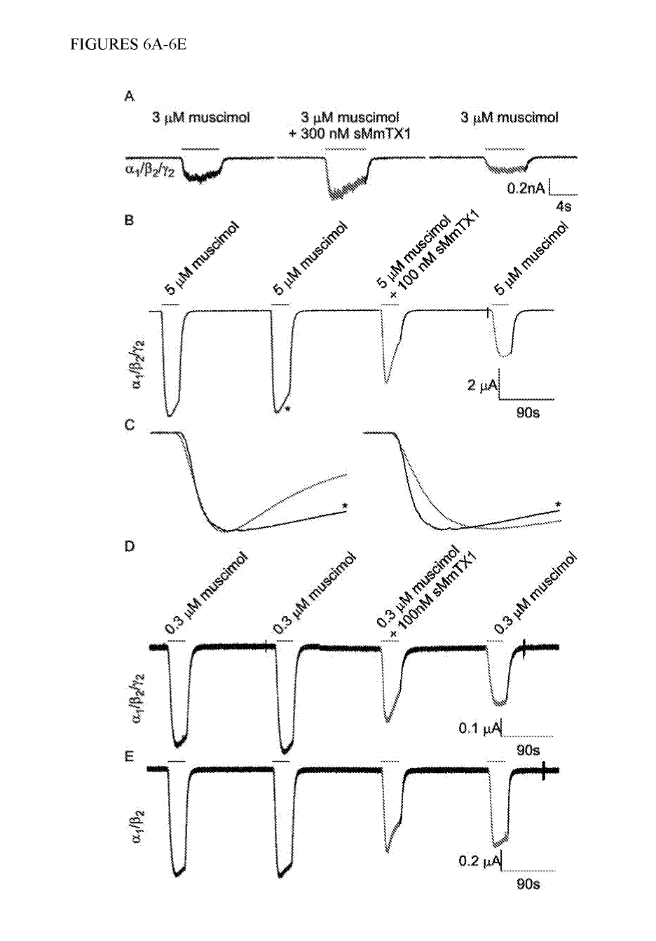

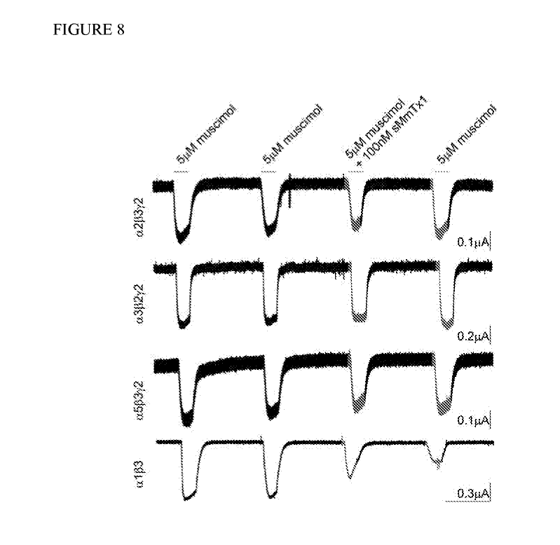

To explore the working mechanism of sMmTX1, we first assessed the effect of the toxin on HEK 293 cells transiently expressing the α1β2γ2 GABA_{A} receptor which is abundantly found in the hippocampus. When applying 0.3 μM muscimol in the presence of 100 nM sMmTX1, we observe an increase in the current of α1β2γ2 receptors by a factor of 2.7±0.18 (n=5). Similar to hippocampal neurons, the current elicited by 50 μM muscimol is not significantly altered by the toxin and as such, the potentiating effect of sMmTX1 is dependent on muscimol concentration. Increasing concentrations of sMmTX1 potentiate the GABA_{A} receptor-mediated current amplitudes elicited by 3 μM muscimol by a similar degree: at 10 nM toxin the current increases by a factor of 1.28±0.12, at 100 nM by 1.33±0.13, and at 300 nM by 1.60±0.11. In addition, we also observe a speeding up of receptor desensitization (FIG. 6A). This trend is also visible when 100 nM rMmTX1 is applied to cultured hippocampal neurons in the presence of 0.3 μM muscimol (FIG. 5B). To examine this phenomenon further, we next expressed the α1β2γ2 GABA_{A} receptor in Xenopus oocytes and assessed receptor desensitization during 30s toxin applications. Taking into account the complex gating mechanisms of rapidly activating GABA_{A} receptors upon agonist application and our relatively slow gravity-fed perfusion system, we did not expect to see a toxin-induced effect on Cl⁻ influx. However, 30s toxin applications may still reveal alterations in receptor desensitization which occur at a slower rate when compared to receptor activation. Therefore, we set up a protocol consisting of two 30s test applications of 5 μM muscimol separated by a 3 minute wash out period. Only when both pulses generated comparable receptor activation, did we proceed with 100 nM sMmTX1 application in concert with 5 μM muscimol. Under control conditions, α1β2γ2 GABA_{A} receptors desensitize slowly in oocytes (single exponential fit yields a time constant (τ) of 48±5s; n=3); however, we observe a substantial increase in receptor desensitization in the presence of 100 nM sMmTX1 (t=25±3s; n=3). As is typical with snake neurotoxins, sMmTX1 washout is very slow and after three minutes, only a fraction of the receptors can be activated again, an observation that fits well with our binding studies (FIG. 2C). When lowering the muscimol concentration to 0.3 μM, we observed a similar effect of 100 nM sMmTX1 on receptor desensitization (FIG. 6D). Interestingly, this outcome is also achieved when expressing only the α1β2-containing receptor in oocytes (FIG. 6E), suggesting that in contrast to benzodiazepines, the γ2 subunit is not required for sMmTX1 efficacy. Moreover, experiments with other subunit compositions (α2β3γ2, α3β2γ2, α5β3γ2, and α1β3) expressed in oocytes suggest an important role for the α1 subunit in determining GABA_{A} receptor susceptibility to sMmTX1 (FIG. 8). As such, a tagged toxin variant may be valuable in identifying al-containing GABA_{A} receptors in heterologous expression systems or tissues. However, future experiments will have to determine whether the α1 subunit is equally important for toxin binding in complex systems since context-dependent variations in pharmacological sensitivity of Cys-loop receptors have been documented.

Altogether, our results with heterologously expressed GABA_{A} receptors suggest that MmTX1 initially increases Cl⁻ influx leading to membrane hyperpolarization. Hereafter, receptor desensitization is promoted which results in a substantial decrease of functioning GABA_{A} receptors. Next, we next determined the effect of sMmTX1 on a neuronal network.

### Example 7

sMmTX1 Influences Spontaneous Hippocampal Neuron Activity.

To assess the influence of sMmTX1 on the activity of a neuronal network, we monitored Ca²⁺ oscillations of hippocampal neurons in culture without and in the presence of toxin. Taking into account the GABA_{A} receptor developmental switch from excitatory to inhibitory, we used cells that were kept in culture for 3 weeks. As such, the transition to inhibitory function should be completed. Cells were incubated in Fluo-4 AM and then subjected to fluorescent time-lapse imaging to measure Ca²⁺ oscillations. Separate applications of 0.3 μM muscimol or 100 nM sMmTX1 have no apparent effect on the frequency of Ca²⁺ spikes (FIGS. 7A, B, D) as compared to cells perfused with extracellular saline solution (98.0±4.8%, n=9 and 105.8±4.0%, n=9, respectively). However, co-application of 0.3 μM muscimol and 100 nM sMmTX1 produces a short but substantial inhibition of Ca²⁺ oscillations, thereby supporting the observation of GABA_{A} receptor activation which in turn inhibits network activity. Remarkably, this inhibitory period is followed by a large increase in the frequency of Ca²⁺ oscillations (171.7±8.4%, n=25 p<0.0001) (FIGS. 7C, D).

Altogether, these results corroborate the hypothesis that sMmTX1 activates and then desensitizes GABA_{A} receptors as evidenced by a decrease followed by an increase in network activity. To further investigate the influence of sMmTX1 on network activity effect, we recorded spontaneous electrophysiological activity in hippocampal neurons. GABA_{A} receptor activation should translate into a decrease in network activity while desensitization should produce an increase in excitability. Cells treated with sMmTX1 show a substantial increase in the frequency of action potentials firing (FIG. 7F; n=5). Interestingly, most of these events occur in bursts which resemble epileptiform activity. Similarly, spontaneous presynaptic release events assessed by voltage-clamp recordings (FIG. 7E; n=5) show a considerable increase in the duration of burst-like postsynaptic currents as compared to mostly single spontaneous events under control conditions. Moreover, each burst is followed by a silence period, as can be clearly seen by an increase in the time between firing (FIG. 7E).

Finally, we examined how these observations compare to the effect of the pore-blocking PTX compound at 100 μM, a commonly used concentration. Although PTX also produces burst-like currents under voltage-clamp recordings (FIG. 7G), the action potential frequency increases more dramatically when compared to sMmTX1. Moreover, these events present themselves as single action potentials or bursts with a much longer duration and a shorter silence interval (FIG. 7G). Altogether, these data support the hypothesis that sMmTX1 acts as an allosteric modulator of GABA_{A} receptors and explain the intermittent seizures observed in animals upon toxin injection into the brain.

### Example 8

Electrophysiological Recording from Xenopus Oocytes.

Each rat GABA_{A} receptor subunit as well as the α1EKβ2γ2 mutant was expressed in Xenopus oocytes from which the vitellin membrane was removed manually. The DNA sequence of all constructs was confirmed by automated DNA sequencing before further usage. cRNA was synthesized using T7 polymerase (Life Technologies) after linearizing the DNA with appropriate restriction enzymes. Channels were expressed in oocytes and currents were studied following 1-2 days incubation after cRNA injection (incubated at 17° C. in 96 mM NaCl, 2 mM KCl, 5 mM HEPES, 1 mM MgCl₂ and 1.8 mM CaCl₂, 50 μg/ml gentamycin, pH 7.6 with NaOH) using two-electrode voltage-clamp recording techniques (OC-725C, Warner Instruments) with a 150 μl recording chamber. Heterologous GABA_{A} receptor manipulations were achieved using previously reported procedures. Microelectrode resistances were 0.5-1 MΩ when filled with 3M KCl. The external recording solution contained (in mM) 100 NaCl, 5 HEPES, 1 MgCl₂ and 1.8 CaCl₂, pH 7.6 with NaOH. All experiments were performed at room temperature (˜22° C.). Off-line data analysis and statistics were performed using Clampfit10 (Molecular Devices), Excel (Microsoft Office) and Origin 8 (OriginLab).

### Example 9

Calcium Imaging of Hippocampal Neurons.

Hippocampi were obtained from Sprague Dawley rat embryos at embryonic day 18, treated with papain (Worthington Biochemical) and dissociated with a pipette. Cells were plated over coverslips coated with Laminin (Life Technologies) and poly-D-Lysine (Sigma-Aldrich). Astrocyte beds were prepared at a density of 80,000 cells/ml, and cultured in DMEM (Life Technologies) with 10% Fetal Bovine Serum, 6 mM glutamine in 5% CO₂ at 37° C. Neurons were plated over 14 days in vitro confluent astrocyte beds at a density of 150,000 cells/ml, and cultured for 3 weeks in Neurobasal supplemented with B27 and 2 mM glutamax. Neurons were incubated with 2 μM Fluo-4 AM (Life Technologies) for 15 minutes. The images were obtained with an Olympus BX51WI microscope equipped with a 40x immersion lens and a Lambda DG-4 ultra-high speed wavelength switcher (Sutter Instruments) using a CCD camera (Hamamatsu) at 2 Hz. The recording chamber was perfused at 2 ml/min at 32° C. with ACSF (Artificial CerebroSpinal Fluid) containing (in mM): 140 NaCl, 5 KCl, 2 CaCl₂, 2 MgCl₂, 10 HEPES, and 10 Glucose (pH 7.3 adjusted with NaOH). Muscimol and sMmTX1 were prepared fresh in recording buffer at 300 nM and 100 nM respectively. Calcium spikes were analyzed with MiniAnalysis (Synaptosoft). Values were expressed as percentage variation on the average frequency of calcium spikes of treated versus control neurons. Data are presented as mean±SEM. Statistical analysis was performed using GraphPad PRISM®. Data was analyzed with paired Student's test.

### Example 10

Creation of sMmTX1-HRK Mutant.

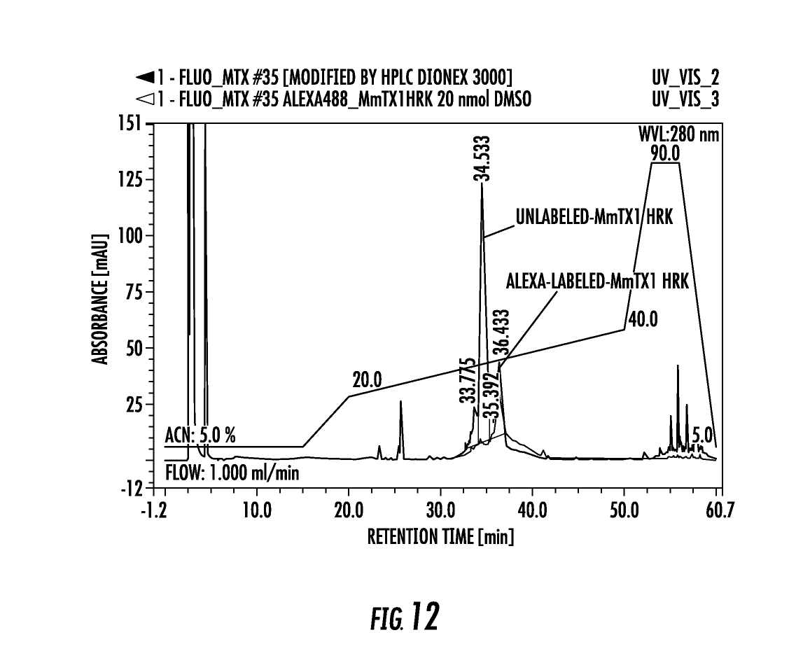

In order to introduce only one reactive primary amine (R—NH₂) per molecule, sMmTX1 was mutated at ⁴His, ²⁸Arg, and ⁴⁷Lys, as well as acetylated at the N-terminus primary amine. Therein, the two Lys residues of sMmTX1 are mutated into ⁴His, ²⁸Arg and ⁴⁷Ala is mutated into ⁴⁷Lys in order to add a single primary amine, which reacts with the Alexa488 TFP (tetrafluorophenyl) ester, an improvement over the succinimidyl ester (SE or NHS-ester) chemistry typically used to attach fluorophores to the primary amines of biomolecules (the acetylated N-terminus prevents reaction with the primary amine at the N-terminus). The sMmTX1-HRK mutant was chemically synthesized using standard solid phase peptide synthesis methodology and Fmoc chemistry. The 5-disulfide bridges were allowed to fold in a 100 mM Tris-HCl buffer, 1 mM EDTA, pH 9.0, adding a combination of GSSG/GSH in a 1 mM/2 mM ratio and sMmTX1 at a concentration of 20 μM. Purification and desalting were achieved by ionic exchange chromatography and C18 HPLC. Typically, the labeling reaction was achieved mixing 20 nmol of sMmTX1-HRK (146 μg) in 20 μl of 100 mM bicarbonate buffer at pH 8.0 with 100 μg in 10 μl of DMSO of Alexa488 TFP ester for 1 hour. Desalting and purification of the final product was achieving by C18 HPLC (FIG. 12). The identity of the final product was checked by mass spectrometry (Alexa-labeled sMmTX1HRK 7857.48 Da; the observed mass difference with sMmTX1 is 517.28 Da and the expected one is 517.23 Da).

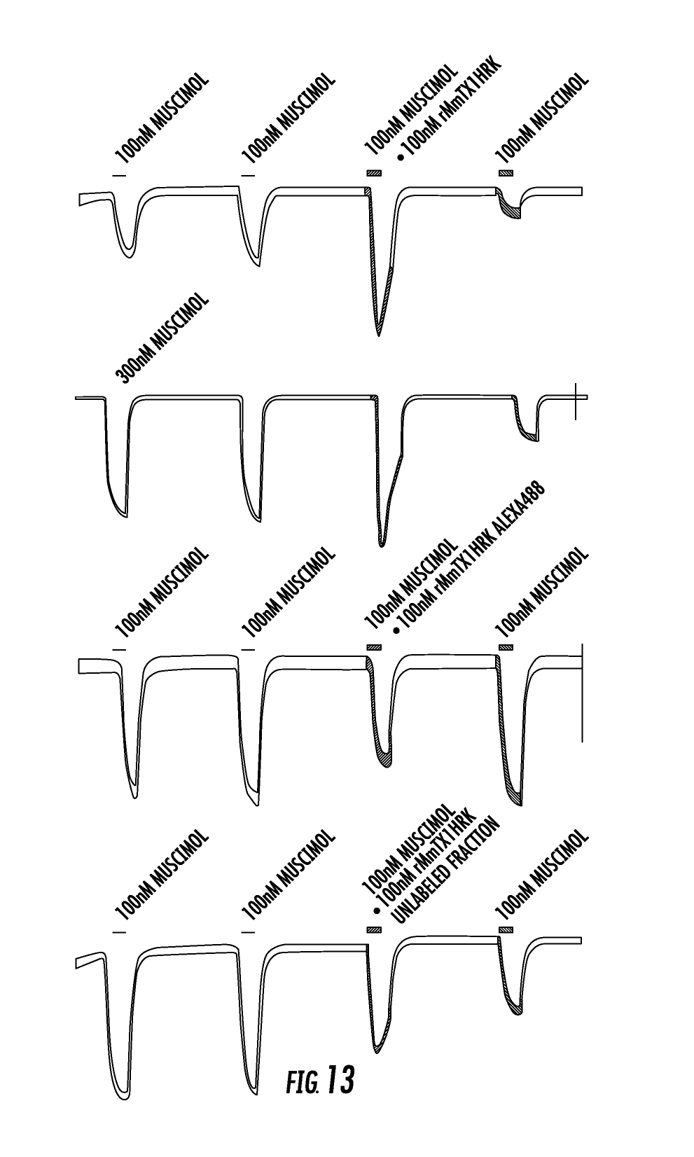

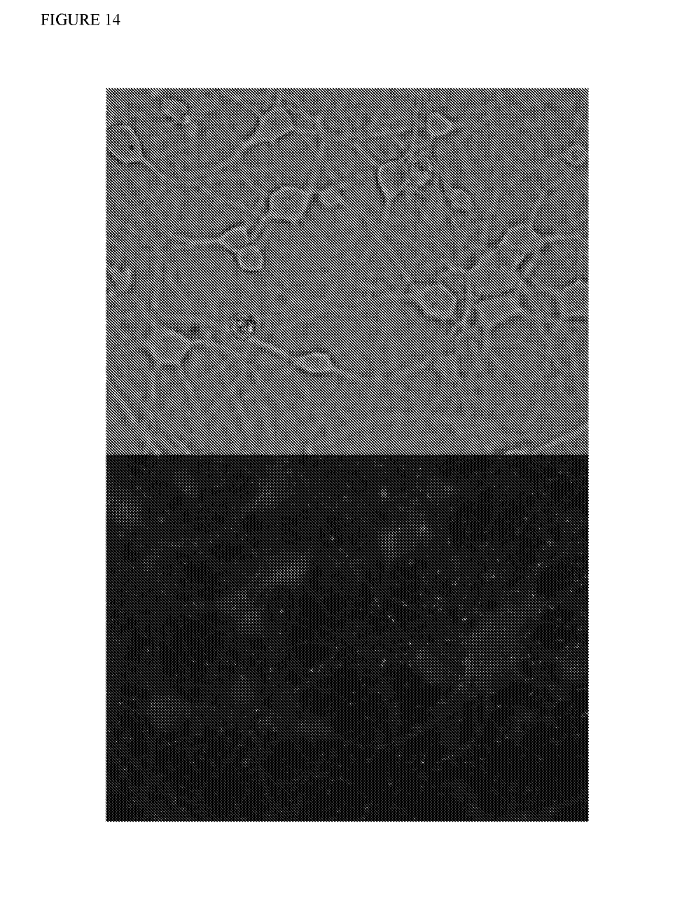

Two 30-s test applications of 100 nM muscimol separated by a 3-minute washout period, followed by 100 nM sMmTX1-HRK-conjugated to Alexa488 application together with 100 nM muscimol was performed. FIG. 13 depicts the effect of the sMmTX1-HRK mutant, sMmTX1-HRK-conjugated to Alexa488, and sMmTX1-HRK unlabeled HPLC fraction on α1β2γ2 GABA_{A} receptor currents. As seen in the top figure, a muscimol concentration of 100 nM activates α1β2γ2 GABA_{A} receptors expressed in Xenopus oocytes held at a membrane voltage of −70 mV. When co-applying 100 nM sMmTX1-HRK, currents are potentiated and receptor desensitization tends to accelerate (red trace). Toxin washout is shown on the right (in green). In the presence of 300 nM muscimol (trace below), the toxin potentiating effect is diminished whereas desensitization effect is unaltered. Expression of the α1β2γ2 receptor in Xenopus oocytes held at a membrane voltage of −70 mV (Middle). The effect of the conjugated toxin on the receptor is reduced but toxin still binds to the receptor (see FIG. 14). In the bottom figure, the same protocol as above is used, but with sMmTX1-HRK unlabeled HPLC fraction on al 2γ2 GABA_{A} receptor currents (see FIG. 12). A representative example of four recordings is shown for all traces.

### Example 11

sMmTX1-HRK Conjugated to Alexa488 Binding to GABA_{A} Receptors in Hippocampal Cells.

sMmTX1-HRK conjugated to Alexa488 was incubated for 45 minutes in culture media together with mouse hippocampal cells (E12 DIV—immature cells). The top figure (FIG. 14) shows neurons under white light whereas the bottom figure shows the same picture under blue light, thereby exciting the Alexa488 fluorophore. Toxin binding to GABA_{A} receptors can be clearly seen as puncta along immature dendrites.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. (canceled)

2. A polypeptide having GABA_{A} modulating activity comprising the following amino acid sequence selected from the group consisting of:
a)
(SEQ ID NO: 1)
LTCKTCPFTTCPNSESCPGGQSICYQRKWEEHRGERIERRCVANCP
AFGSHDTSLLCCTRDNCN;
(SEQ ID NO: 2)
LTCKTCPFTTCPNSESCPGGQSICYQRKWEEHHGERIERRCVANCP
AFGSHDTSLLCCTRDNCN;
(SEQ ID NO: 8)
Leu Thr Cys Lys Thr Cys Pro Phe Thr Thr Cys Pro
Asn Ser Glu Ser Cys Pro Gly Gly Gln Ser Ile Cys
Tyr Gln Arg Lys Trp Glu Glu His Arg Gly Glu Arg
Ile Glu Arg Arg Cys Val Ala Asn Cys Pro Ala Phe
Gly Ser His Asp Thr Leu Leu Cys Cys Thr Arg Asp
Asn Cys Asn;
(SEQ ID NO: 9)
Met Lys Cys Leu Ile Cys Pro Phe Thr Thr Cys Ser
Gln Ser Glu Ser Cys Pro Gly Gly Gln Ser Ile Cys
Phe Gln Arg Lys Phe Asp Asp Arg His Gly Asp Arg
Ile Glu Arg Gly Cys Ala Val Thr Cys Pro Pro Phe
Gly Ser His Asp Thr Ile Phe Cys Cys Ser Thr Asn
Asp Cys Asn;
(SEQ ID NO: 10)
Ile Glu Cys His Asn Cys Pro Phe Thr Thr Cys Gly
Asn Ser Glu Ser Cys Pro Gly Gly Gln Ser Ile Cys
Val Gln Arg Lys Leu Glu Glu Lys Lys Gly Glu Arg
Ile Glu Arg Ser Cys Thr Asp Gly Cys Pro Gly Phe
Gly Ser His Asp Thr Val Glu Cys Cys Arg Ile Ala
Arg Cys Asn;
(SEQ ID NO: 11)
Arg Gln Cys Tyr Thr Cys Pro Phe Thr Thr Cys His
Asn Ser Glu Ser Cys Pro Gly Gly Gln Ser Ile Cys
Tyr Gln Arg Lys Tyr Glu Glu His Arg Gly Glu Arg
Ile Glu Arg Lys Cys Ser Leu Ser Cys Pro Ser Phe
Gly Ser His Asp Thr Leu Leu Cys Cys Ala Arg Pro
Lys Cys Asn;
(SEQ ID NO: 12)
Phe Arg Cys Phe Arg Cys Pro Phe Thr Thr Cys Asn
Asn Ser Glu Ser Cys Pro Gly Gly Gln Ser Ile Cys
Tyr Gln Arg Lys Trp Glu Glu His Arg Gly Glu Arg
Ile Glu Arg Arg Cys Val Ala Asn Cys Pro Ala Phe
Gly Ser His Asp Thr Leu Leu Cys Cys Lys Arg Glu
Glu Cys Asn;
(SEQ ID NO: 13)
Leu Ser Cys Asn Thr Cys Pro Phe Thr Thr Cys Gln
Asn Ser Glu Ser Cys Pro Gly Gly Gln Ser Ile Cys
Tyr Gln Arg Lys Trp Glu Glu His Arg Gly Glu Arg
Ile Glu Arg Arg Cys Val Ala Asn Cys Pro Ala Phe
Gly Ser His Asp Thr Leu Leu Cys Cys Thr Arg Asp
Asn Cys Asn;
and
(SEQ ID NO: 14)
Leu Leu Cys Lys Thr Cys Pro Phe Thr Thr Cys Pro
Asn Ser Glu Ser Cys Pro Gly Gly Gln Ser Ile Cys
Tyr Gln Arg Lys Trp Glu Glu His Arg Gly Glu Arg
Ile Glu Arg Arg Cys Val Ala Asn Cys Pro Ala Phe
Gly Ser His Asp Thr Leu Leu Cys Cys Thr Arg Asp
Asn Cys Asn;
b) a functional fragment of any of the peptides of a);
c) a functional homolog of any of the peptides of a) or b) or functional fragment thereof; and
d) a fusion polypeptide comprising an amino acid sequence of any of any of the peptides of a) to c).

3. (canceled)

4. A sMmTx1-HRK mutant polypeptide having GABA_{A} modulating activity comprising the following amino acid sequence with an acetyl functional group at the N-terminus (Acl):
a)
(SEQ ID NO: 21)
(Acl)LTCHTCPFTTCPNSESCPGGQSICYQRRWEEHRGERIERRCVANC
PKFGSHDTSLLCCTRDNCN;
b) a functional fragment of a);
c) a functional homolog of a) or b) or functional fragment thereof; and
d) a fusion polypeptide comprising an amino acid sequence of any of a) to c).

5. A polypeptide having GABA_{A} modulating activity comprising the following amino acid sequence:
a)
(SEQ ID NO: 6)
Xaa Xaa Cys Lys Thr Cys Pro Phe Thr Thr Cys Pro
Asn Ser Glu Ser Cys Xaa Xaa Xaa Xaa Xaa Xaa Cys
Tyr Gln Arg Lys Trp Glu Glu His Arg Gly Glu Arg
Ile Glu Arg Arg Cys Xaa Xaa Xaa Cys Pro Ala Phe
Gly Ser His Asp Thr Ser Xaa Xaa Cys Cys Thr Arg
Asp Asn Cys Asn;
b) a functional fragment of a);
c) a functional homolog of a) or b) or functional fragment thereof; and
d) a fusion polypeptide comprising an amino acid sequence of any of a) to c).

6. A polypeptide having GABA_{A} modulating activity comprising the following amino acid sequence:
a)
(SEQ ID NO: 7)
Xaa Xaa Cys Lys Thr Cys Pro Phe Thr Thr Cys Pro
Asn Ser Glu Ser Cys Xaa Xaa Xaa Xaa Xaa Xaa Cys
Tyr Gln Arg Lys Trp Glu Glu His His Gly Glu Arg
Ile Glu Arg Arg Cys Xaa Xaa Xaa Cys Pro Ala Phe
Gly Ser His Asp Thr Ser Xaa Xaa Cys Cys Thr Arg
Asp Asn Cys Asn;
b) a functional fragment of a);
c) a functional homolog of a) or b) or functional fragment thereof; and
d) a fusion polypeptide comprising an amino acid sequence of any of a) to c).

7.-13. (canceled)

14. A nucleic acid sequence encoding any of the polypeptides having GABA_{A} modulating activity or derivatives, homologues, analogues or mimetics thereof of claim 2.

15. A vector comprising one or more nucleic acid sequences encoding any of the polypeptides having GABA_{A} modulating activity or derivatives, homologues, analogues or mimetics thereof claim 2.

16. A composition comprising one or more polypeptides having GABA_{A} modulating activity claim 2, and at least one or more biologically active agents.

17. A composition comprising one or more polypeptides having GABA_{A} modulating activity claim 2, and at least one or more imaging agents.

18.-19. (canceled)

20. A method for modulating GABA_{A} receptors in a subject suffering from a neurological disorder comprising administering to the subject an effective amount of a composition comprising one or more polypeptides having GABA_{A} modulating activity of claim 2.

21. The method of claim 20, wherein the method further comprises administering to the subject an effective amount of at least one or more biologically active agents.

22. A method for imaging GABA_{A} receptors in a subject suffering from a neurological disorder comprising administering to the subject an effective amount of a composition comprising one or more polypeptides having GABA_{A} modulating activity of claim 2, and at least one or more imaging agents.


SEQUENCE LISTING (21 sequences)

SEQ ID NO: 1 - protein, 64 aa, Micrurus mipartitus
LTCKTCPFTTCPNSESCPGGQSICYQRKWEEHRGERIERRCVANCPAFGSHDTSLLCCTRDNCN

SEQ ID NO: 2 - protein, 64 aa, Micrurus mipartitus
LTCKTCPFTTCPNSESCPGGQSICYQRKWEEHHGERIERRCVANCPAFGSHDTSLLCCTRDNCN

SEQ ID NO: 3 - protein, 15 aa, Artificial Sequence
  synthetic sequence
CKTCPFTTCPNSESC

SEQ ID NO: 4 - protein, 18 aa, Artificial Sequence
  synthetic sequence
CYQRKWEEHRGERIERRC

SEQ ID NO: 5 - protein, 18 aa, Artificial Sequence
  synthetic sequence
CYQRKWEERRGERIERRC

SEQ ID NO: 6 - protein, 64 aa, Artificial Sequence
  synthetic sequence
  MISC_FEATURE: X can be any natural or unnatural amino acid
  MISC_FEATURE (1)..(2): X can be any natural or unnatural amino acid
  MISC_FEATURE (18)..(23): X can be any natural or unnatural amino acid
  MISC_FEATURE (42)..(44): X can be any natural or unnatural amino acid
  MISC_FEATURE (55)..(56): X can be any natural or unnatural amino acid
XXCKTCPFTTCPNSESCXXXXXXCYQRKWEEHRGERIERRCXXXCPAFGSHDTSXXCCTRDNCN

SEQ ID NO: 7 - protein, 64 aa, Artificial Sequence
  synthetic sequence
  MISC_FEATURE (1)..(2): X can be any natural or nonnatural amino acid
  MISC_FEATURE (18)..(23): X can be any natural or nonnatural amino acid
  MISC_FEATURE (42)..(44): X can be any natural or nonnatural amino acid
  MISC_FEATURE (55)..(56): X can be any natural or nonnatural amino acid
XXCKTCPFTTCPNSESCXXXXXXCYQRKWEEHHGERIERRCXXXCPAFGSHDTSXXCCTRDNCN

SEQ ID NO: 8 - protein, 63 aa, Artificial Sequence
  synthetic sequence
LTCKTCPFTTCPNSESCPGGQSICYQRKWEEHRGERIERRCVANCPAFGSHDTLLCCTRDNCN

SEQ ID NO: 9 - protein, 63 aa, Artificial Sequence
  synthetic sequence
MKCLICPFTTCSQSESCPGGQSICFQRKFDDRHGDRIERGCAVTCPPFGSHDTIFCCSTNDCN

SEQ ID NO: 10 - protein, 63 aa, Artificial Sequence
  synthetic sequence
IECHNCPFTTCGNSESCPGGQSICVQRKLEEKKGERIERSCTDGCPGFGSHDTVECCRIARCN

SEQ ID NO: 11 - protein, 63 aa, Artificial Sequence
  synthetic sequence
RQCYTCPFTTCHNSESCPGGQSICYQRKYEEHRGERIERKCSLSCPSFGSHDTLLCCARPKCN

SEQ ID NO: 12 - protein, 63 aa, Artificial Sequence
  Synthetic sequence
FRCFRCPFTTCNNSESCPGGQSICYQRKWEEHRGERIERRCVANCPAFGSHDTLLCCKREECN

SEQ ID NO: 13 - protein, 63 aa, Artificial Sequence
  Synthetic sequence
LSCNTCPFTTCQNSESCPGGQSICYQRKWEEHRGERIERRCVANCPAFGSHDTLLCCTRDNCN

SEQ ID NO: 14 - protein, 63 aa, Artificial Sequence
  Synthetic sequence
LLCKTCPFTTCPNSESCPGGQSICYQRKWEEHRGERIERRCVANCPAFGSHDTLLCCTRDNCN

SEQ ID NO: 15 - protein, 47 aa, Rattus norvegicus
DGSRLNQYDLLGQTVDSGIVQSSTGEYVVMTTHFHLKRKIGYFVIQT

SEQ ID NO: 16 - protein, 47 aa, Rattus norvegicus
DGSRLNQYDLLGQSIGKETIKSSTGEYTVMTAHFHLKRKIGYFVIQT

SEQ ID NO: 17 - protein, 47 aa, Rattus norvegicus
DGSRLNQYDLLGHVVGTEIIRSSTGEYVVMTTHFHLKRKIGYFVIQT

SEQ ID NO: 18 - protein, 47 aa, Rattus norvegicus
ESSSLVQYDLIGQTVSSETIKSITGEYIVMTVYFHLRRKMGYFMIQT

SEQ ID NO: 19 - protein, 47 aa, Rattus norvegicus
DGSRLNQYHLMGQTVGTENISTSTGEYTIMTAHFHLKRKIGYFVIQT

SEQ ID NO: 20 - protein, 47 aa, Rattus norvegicus
ESSSLLQYDLIGQTVSSETIKSNTGEYVIMTVYFHLQRKMGYFMIQI

SEQ ID NO: 21 - protein, 64 aa, Artificial Sequence
  synthetic sequence
LTCHTCPFTTCPNSESCPGGQSICYQRRWEEHRGERIERRCVANCPKFGSHDTSLLCCTRDNCN